(19) United States
(12) Patent Application Publication
(10) Pub. No.: US 2023/0243773 A1
(43) Pub. Date: Aug. 3, 2023

(54) Power Transfer-Based Flexible Sensing System for Smart Hydrogel Swelling State Readout

(71) Applicant: University of Utah Research Foundation, Salt Lake City, UT (US)

(72) Inventors: Christopher F. Reiche, Salt Lake City, UT (US); Julia Koerner, Hannover (DE); Benozir Ahmed, Salt Lake City, UT (US); Florian Solzbacher, Salt Lake City, UT (US); Jules J. Magda, Salt Lake City, UT (US)

(21) Appl. No.: 18/161,697

(22) Filed: Jan. 30, 2023

Related U.S. Application Data
(60) Provisional application No. 63/304,428, filed on Jan. 28, 2022.

(51) Int. Cl. G01N 27/22; G01N 27/12; A61B 5/145

(52) U.S. Cl. CPC G01N 27/227; G01N 27/126; A61B 5/14546; A61B 5/14539; A61B 2562/12

(57) ABSTRACT

A power transfer-based flexible sensing platform that can be used in a fluid environment includes at least one metallic thin film structure, with a polymeric encapsulation that protects the metallic thin film structure from the fluid environment, a smart hydrogel sandwiched between layers of the metallic thin film structure, the smart hydrogel being configured to swell or shrink in response to stimulus, and an AC power source or signal generator electrically connected to the at least one metallic film structure. The metallic thin film can include first and second U-shaped metallic thin film structures, each encapsulated, where the smart hydrogel is sandwiched between them. An AC power or signal source is electrically connected to one of the U-shaped structures, causing power to be inductively transferred to the other. Measurement of the induced power or other signal can be used to determine a concentration of an analyte in the fluid environment.

### CROSS-REFERENCE TO RELATED APPLICATIONS

The present application claims the benefit under 35 U.S.C. 119(e) of U.S. Application No. 63/304,428, filed Jan. 28, 2022, entitled POWER TRANSFER-BASED FLEXIBLE SENSING SYSTEM FOR SMART HYDROGEL SWELLING STATE READOUT, which is herein incorporated by reference in its entirety.

### STATEMENT AS TO FEDERALLY-SPONSORED RESEARCH

This invention was made with government support under GM130241 awarded by the National Institutes of Health. The government has certain rights in the invention.

### BACKGROUND

Hydrogel materials are three-dimensionally crosslinked polymer networks that absorb liquid and change their swelling state depending on surrounding environmental factors, e.g., pH, temperature, ionic concentration, and/or light. Hydrogels can be made “smart” (stimuli-responsive) to respond to the presence of a desired analyte, by functionalizing with chemical moieties, analyte-specific aptamers or molecular imprinting. Their potential for good biocompatibility and their selective analyte recognition capability make these polymers a promising candidate for biosensors. However, it can be challenging to detect the hydrogel's swelling state. Any proposed practical solutions need to take the individual requirements of the target application into account.

Under current practice, analyte blood concentrations during surgery or the like is monitored by drawing blood and sending the sample to a centralized laboratory or other localized analysis stations with the results available only after a significant period of time. However, most critical incidents caused by drug or other target molecule levels being too high or too low need to be resolved quickly, and thus continuous monitoring of these levels would be an important step to improving patient outcomes and saving lives.

### BRIEF SUMMARY

The present invention provides for a readout method with corresponding devices that can be used for acute monitoring of bio-analyte levels during surgery or the like, where the device is mounted to a catheter. Additionally, the presently disclosed concepts can have application in other cases of biomedical sensing. The invention can also be adapted to other biomedical sensing purposes for continuous monitoring of analytes for which often no existing solutions are available. In addition to use in monitoring analyte levels in a biomedical environment, it will be appreciated that such devices and methods could also be used in other environments (e.g., including gaseous environments) and situations where monitoring of a given analyte level is desired (e.g., pipeline monitoring, etc.).

In order to detect the swelling state of a hydrogel, a power and/or or signal transfer-based flexible sensing platform was designed. The sensing platform incorporates a metallic thin film structure with a biocompatible polymeric encapsulation which protects the thin metal film from the physiological fluid or other environment. In an embodiment, two such films can be connected back-to-back with a medical-grade adhesive with an optional conductive layer (e.g., aluminum) acting as electronic shielding in-between portions of such structures. A smart hydrogel can be sandwiched in-between the films, which hydrogel swells or shrinks in response to the stimulus, e.g., analyte concentration, in the physiological fluid or other fluid being sampled. One of the metallic structures is electrically connected to a power or signal source (e.g., through the catheter), while the power or signal transferred (e.g., by induction) to the other metallic structure can be read out. The swelling or shrinking of the hydrogel changes the distance and/or angle of orientation between the two metallic films. The power and/or signal transferred between the metal layers varies with these changes as well, and this change can be utilized to calculate the analyte concentration, based on a reference correlation (e.g., determined during calibration). By way of example, platinum (Pt) or another noble metal can be used to fabricate the thin metallic structure and polyimide (PI) or another biocompatible polymer can be used as the encapsulation material. In an aspect of the present invention, a medical grade epoxy can be used as a biocompatible adhesive to attach the structures to one another.

The present invention has several benefits or aspects that provide advantages in that the sensor is made of biocompatible materials, it can be very thin and flexible, hence eliciting a reduced foreign body response and potentially allowing for a variety of applications in different media. Furthermore, the sensor is easily adaptable for different analytes as the smart hydrogel can be tailored as needed, which will, however, not influence the readout principle of the swelling state. The power transfer-based readout employs a very small amount of heat generation due to Joule heating and does not induce any electrolysis within the sample fluid. Unlike optical or fluorescent-based biomarker sensors, the proposed sensor can advantageously be successfully employed in an opaque medium. It is anticipated that the protective encapsulation layer over the metallic structure will offer extended longevity, and the sensor will be durable, so as to be suitable for continuous, long-term biomarker monitoring.

According to an embodiment, a power transfer-based flexible sensing platform as described may include at least one metallic thin film structure, the metallic thin film structure having a polymeric encapsulation that protects the metallic thin film structure from the fluid environment in which it is deployed. A smart hydrogel may be sandwiched between layers of the encapsulated metallic thin film structure, where the smart hydrogel structure is configured to swell or shrink in response to stimulus. A power source may be electrically connected to the at least one metallic film structure. Additionally, read out electronic or other equipment may be electrically connected to the at least one metallic film structure, for read out. In a typical embodiment, the at least one metallic film structure may include two such thin metallic films, separated from one another by the smart hydrogel. Another embodiment that still operates on such a power transfer/induction principle could include an encapsulated metallic thin film structure, with two electrically separated structures, wrapped around the hydrogel. Swelling or shrinking of the hydrogel would change the separation distance between such structures, altering the induced signal that can be read out. Another embodiment that includes a single encapsulated metallic thin film structure with an attached hydrogel that bends upon swelling or shrinking of the hydrogel is also described herein.

In an embodiment, the fluid environment is a physiological fluid environment.

In an embodiment, the stimulus that the smart hydrogel swells or shrinks in response to includes at least one of pH, temperature, concentration of an analyte, or light.

In an embodiment, the stimulus that the smart hydrogel swells or shrinks in response to is an analyte concentration, which analyte is present in the fluid environment.

In an embodiment, the analyte comprises at least one of pH or glucose concentration of the fluid in the fluid environment.

In an embodiment, the analyte concentration comprises a concentration of a drug in the fluid environment.

In an embodiment, the metallic thin film structure comprises a noble metal.

In an embodiment, the metallic thin film structure comprises platinum.

In an embodiment, the polymeric encapsulation comprises a polyimide.

In an embodiment, the hydrogel is functionalized with one or more chemical moieties, an analyte-specific aptamer or molecular imprinting to be sensitive to a specifically selected analyte.

In an embodiment, the smart hydrogel is present at a distal tip of the sensing platform.

In an embodiment the sensing platform further comprises an enclosure with a membrane around the distal tip including the smart hydrogel.

In an embodiment, the enclosure includes electrical shielding.

In an embodiment, the membrane around the distal tip is configured to transduce an analyte concentration of the fluid environment outside of the enclosure to another analyte concentration inside of the enclosure.

In an embodiment, the sensing platform transduces an analyte concentration in the fluid environment outside of the enclosure to a pH change within the enclosure, the smart hydrogel inside the enclosure being configured to swell or shrink in response to the change in pH.

In an embodiment, the power source applies an alternating current (AC) to the at least one metallic film structure.

In an embodiment, the alternating current has a frequency of 5 to 100 MHz, or 10 to 50 MHz.

In an embodiment, the polyimide or other polymeric encapsulation has a thickness of less than 500, less than 400, less than 300, less than 200, less than 150, less than 100, less than 50, or less than 10 μm (e.g., 1-10 μm).

In an embodiment, the thin metal film layers have a thickness of less than 1000, less than 800, less than 600, or less than 500 nm (e.g., 50-500 nm, or 100-500 nm).

In an embodiment, the hydrogel at the tip of the device has a thickness of less than 1000, less than 800, less than 600, or less than 500 μm (e.g., 100-500 μm, or 250-450 μm).

In an embodiment, the at least one metallic thin film structure comprises a pair of U-shaped metallic thin film structures, each encapsulated in a polymeric encapsulation, wherein the smart hydrogel is sandwiched between the pair of U-shaped metallic thin film structures (e.g., at the tip thereof), the smart hydrogel being configured to swell or shrink in response to the stimulus, wherein the power source is electrically connected to a first U-shaped metallic thin film structure of the pair of U-shaped metallic thin film structures, so as to cause power and/or signal to be transferred from the first U-shaped metallic thin film structure to a second U-shaped metallic thin film structure of the pair of U-shaped metallic thin film structures by induction when the power source is activated, measurement of the induced power signal in the second-U-shaped metallic thin film structure being used to determine a concentration of an analyte in the fluid environment. Additionally, read out electronic or other equipment may be electrically connected to the at least one metallic film structure (e.g., to the unpowered structure), for read out.

According to another embodiment, a power transfer-based flexible sensing platform used in a physiological fluid environment includes at least one metallic thin film structure, the metallic thin film structure having a biocompatible polymeric encapsulation that protects the metallic thin film structure from the physiological fluid. A smart hydrogel is sandwiched between layers of the metallic thin film structure (e.g., at the tip thereof), the smart hydrogel being configured to swell or shrink in response to stimulus, and a power source is electrically connected to the at least one metallic film structure. Additionally, read out electronic or other equipment may be electrically connected to the at least one metallic film structure, for read out.

While the described embodiments generally include sandwiching of the smart hydrogel between two layers of a metallic thin film structure, a simpler sensor device is also possible, although the device with the sandwiched smart hydrogel provides distinct advantages. According to such an alternative simplified embodiment, an impedimetric flexible sensing platform used in a fluid environment can include just one metallic thin film structure, the metallic thin film structure having a polymeric encapsulation that protects the metallic thin film structure from the fluid environment. A smart hydrogel is attached to the metallic thin film structure, the smart hydrogel being configured to swell or shrink in response to stimulus, so as to cause the metallic thin film structure to bend at a different angle when the stimulus is present, versus when the stimulus is not present. A power source is electrically connected to the at least one metallic film structure. Additionally, read out electronic or other equipment may be electrically connected to the metallic thin film structure, for read out. The bending deformation of the structure causes alterations in the impedance, which can be monitored and calibrated to transduce the concentration of the analyte present in the fluid medium.

In a single metal layer embodiment, swelling or shrinking of the hydrogel causes the metallic thin film structure to bend at a different angle when the stimulus is present, versus when the stimulus is not present, changing the impedance of the metal structure. The change in impedance or other signal associated with the thin metal film structure can be correlated, and “read out” to determine analyte concentration within the fluid environment.

In any of the sandwiched hydrogel embodiments, swelling or shrinking of the hydrogel causes the separation distance between the two metallic layers to change. This change in distance changes the strength of the induced power or signal transferred from the powered metallic layer, to the other metallic layer, and this change in power or other signal can be correlated, and “read out” to determine analyte concentration within the fluid environment.

As used herein, power and/or signal transfer is meant to describe any type of electromagnetic field-based coupling between two electrically separated metallic thin film structures. For example, in an embodiment, the power and/or signal may be transferred from the powered metallic thin film structure, to the other metallic thin film structure, due to induction.

Features from any of the disclosed embodiments may be used in combination with one another, without limitation. This summary is provided to introduce a selection of concepts in a simplified form that are further described below in the detailed description. This summary is not necessarily intended to identify key or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the components and combinations particularly pointed out in the appended claims. These and other features of the present disclosure will become more fully apparent from the following description and appended claims, or may be learned by the practice of the disclosure as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the drawings located in the specification. It is appreciated that these drawings depict only typical embodiments of the invention and are therefore not to be considered limiting of its scope. The invention will be described and explained with additional specificity and detail through the use of the accompanying drawings.

FIG. 1 shows a schematic of an exemplary sensor structure before hydrogel attachment that may include just a single encapsulated metallic layer.

FIG. 1A shows a schematic drawing of a completed exemplary sensor structure including two encapsulated metallic layers (with the tip including a sandwiched hydrogel shown in the inset).

FIG. 2A shows a schematic of the exemplary single metallic layer sensor of FIG. 1 including the hydrogel, before bending.

FIG. 2B shows a schematic of the exemplary single metallic layer sensor of FIG. 1 including the hydrogel, after bending.

FIG. 3 shows an equivalent circuit model of the sensor of FIG. 1 and FIGS. 2A-2B with L_{S}, R_{S}, C_{S} representing the sensor inductance, resistance, and capacitance, respectively (framed part); C_{P1} presents the sensor-to-ground capacitance, and L and C_{P2} are the connection cable inductance and capacitance; and R_{P} is the sensor-to-solution resistance.

FIG. 3A shows a lumped element equivalent circuit model for an exemplary thin film bending sensor with attached hydrogel (e.g., such as that shown in FIG. 1); L_{S}, R_{S}, and C_{S} represent the sensor inductance, resistance, and capacitance respectively (framed part); C_{P1} is the sensor-to-ground capacitance, and L and C_{P2} are the connection cable inductance and capacitance; R_{P} is the sensor-to-solution resistance.

FIG. 4 shows an employed experimental setup, where the sensor is placed in an L-shaped solution container and connected to a network analyzer with shielded cables.

FIG. 5 shows experimentally measured sensor impedance magnitude in DI water, air, and 1×PBS immediately after the introduction of solution and 1×PBS after two hours of exposure to the solution (allowing for hydrogel saturation and, hence the completion of sensor bending).

FIGS. 6A-6D show impedance magnitude in air (FIG. 6A), DI water (FIG. 6B), 1×PBS at time t=0 h (FIG. 6C), and 1×PBS at time t=2 h (FIG. 6D) compared with circuit simulation results.

FIGS. 7A-7H show resistive impedance in air (FIG. 7A), DI water (FIG. 7B), 1×PBS at time t=0 h (FIG. 7C), 1×PBS at time t=2 h (FIG. 7D), and reactive impedance in air (FIG. 7E), DI water (FIG. 7F), 1×PBS at time t=0 h (FIG. 7G), and 1×PBS at time t=2 h (FIG. 7H).

FIGS. 8A-8F show schematics of an exemplary polyimide encapsulated platinum structure fabrication process. Such process principles may be used in formation of embodiments including a single metallic layer, as well as dual layer embodiments as described herein (by simply attaching two such structure together, back-to-back). FIG. 8A shows liquid polyimide precursor is spin-coated on the carrier substrate (e.g., silicon wafer) and baked to solidify. FIG. 8B shows patterning of photoresist (PR) for definition of metal traces. FIG. 8C shows the platinum structure generated by sputter deposition and a subsequent lift-off process. FIG. 8D shows another layer of polyimide is deposited and baked to encapsulate the platinum structure. FIG. 8E shows deposition and patterning of photoresist to use as etch mask. FIG. 8F shows dry etching of the polyimide to access the platinum bond pad(s).

FIGS. 9A-9D show schematics of cable connection and hydrogel polymerization on a microfabricated polyimide encapsulated single layer platinum structure. Similar process principles may be used in formation of embodiments including a dual layer embodiment. FIG. 9A shows the polyimide encapsulated platinum structure is attached to a polylactic acid (PLA) substrate with epoxy (except the tip) as support structure. FIG. 9B shows cables for electrical connection are soldered to the bondpad and encapsulated with epoxy. FIG. 9C shows a 400 μm smart hydrogel is UV polymerized from pregel solution on the polyimide tip with the help of a mold and 2 glass slides to define the desired shape. FIG. 9D shows the glass slide and mold are removed from the sensor after polymerization.

FIG. 10 shows output voltage of the receiving sensor part for the hydrogel response to alternating 0 mM and 5 mM glucose (with phosphate buffered saline (PBS) as carrier solution) measured by a Zurich instruments lock-in amplifier. For the transmitter input signal, a sine wave of 25 MHz frequency and 100 mV peak-to-peak voltage was used.

FIG. 11 shows a schematic sketch of a sensor as fabricated (top) and bent in response to the smart hydrogel's volume change (bottom).

FIGS. 12A-12B show relation between mechanical parameters of sensor dummy structures. FIG. 12A shows calculated spring constants dependent on polyimide thickness (see eq. (1)). FIG. 12B shows resulting bending angle change for different hydrogel thicknesses and spring constants. To induce the bending, the mechanical sensor dummies were immersed in ⅓× and 1×PBS for 30 minutes each.

FIGS. 13A-13B show results for a repeated bending stability test of a 400 μm hydrogel on a 6.3 μm thick polyimide sheet. FIG. 13A shows absolute bending angles for each concentration step. FIG. 13B shows change of bending angle within each solution cycle from ⅓×PBS to 1×PBS (30 minutes immersion time for each).

FIG. 14 schematically shows hydrogel shrinking in response to different stimuli.

FIG. 15 shows a schematic of a single polyimide sheet with an encapsulated U-shaped platinum structure, with dimensions provided in μm.

All Figures from FIG. 16 onward relate to embodiments including two metallic thin film layers, which are electrically isolated from one another, where power or another signal provided to the first layer induces a transferred power or signal within the other metallic layer, and which can be read out, to determine the analyte concentration. FIG. 16 shows a schematic of an exemplary sensor structure including two metallic thin film layers, including a zoomed-in exploded view shown in the inset.

FIG. 17 shows results of an in-vitro functionality test of power transfer based smart hydrogel PI sensors for solution exchange between 1×PBS (pH 7.4) and 1×PBS (pH 7.7).

FIG. 18 shows results of an in-vitro step test of power transfer based smart hydrogel PI sensors for solution exchange between 1×PBS (pH 7.4) and 1×PBS (pH 8.2) using a pH step in 0.2 increments.

FIG. 19 shows a schematic of a single long polyimide sheet with encapsulated U-shaped platinum structure (dimensions provided in μm).

FIG. 20 shows results of an in-vitro functionality test of power transfer based smart hydrogel PI sensors for solution exchange between 1×PBS and 5 mM Glucose.

FIG. 21 shows results of an in-vitro resetting test of power transfer based smart hydrogel PI sensors for solution exchange between 3 mM, 6 mM, and 9 mM glucose with a reset step of 1×PBS between glucose steps.

FIG. 22 shows another circuit model for power transfer based smart hydrogel PI sensors.

FIGS. 23A-23F show schematic illustrations of another polyimide encapsulated platinum structure fabrication process. FIG. 23A shows how liquid polyimide is spin-coated on the carrier substrate (silicon) and baked to solidify. FIG. 23B shows patterning of photoresist for the definition of metal traces. FIG. 23C shows how the platinum structure is generated by sputter deposition and a subsequent lift-off process. FIG. 23D shows how another layer of polyimide is deposited to encapsulate the platinum structure. FIG. 23E shows deposition and patterning of photoresist to use as etch mask. FIG. 23F shows dry etching of the polyimide to access the platinum bond pads.

FIGS. 24A-24H show schematics of a power transfer-based smart hydrogel sensor fabrication process. The platinum traces within the polyimide layers are not shown, due to size, and for simplicity, although it is appreciated that they are present, as detailed in FIGS. 23A-23F. FIG. 24A shows how two polyimide (PI) encapsulated metal structures are connected back-to-back with an aluminum sheet in between. FIG. 24B shows how cables are connected to the bond pad, encapsulated with medical-grade epoxy, and then the resulting structure is covered with an aluminum shield except for the sensor tip. FIG. 24C shows how the upper PI sheet is bent and kept folded with the adhesive force of a water droplet while the lower PI is placed on a glass slide to keep it flat. FIG. 24D shows how hydrogel pre gel solution is deposited in a PLA mold (spacer). FIG. 24E shows how a glass slide is used on the mold to achieve the desired thickness of the hydrogel which should ideally be the same as the thickness of the spacer. FIG. 24F shows how the pre-gel solution is exposed to UV light to polymerize into a hydrogel. FIG. 24G shows release of the folded PI sheet to touch the hydrogel and UV light exposure from sides to connect with the hydrogel. FIG. 24H shows how the sensor is removed from the glass surface.

FIG. 25 shows a schematic of the experimental setup used for the in-vitro characterization of the power transfer-based PI sensor. The setup includes a syringe pump to withdraw solution for a continuous solution exchange. A lock-in amplifier was used to record the sensor response at a specific frequency.

FIGS. 26A-26B show results of the in-vitro functionality test of power transfer based smart hydrogel PI sensors. FIG. 26A shows results for solution exchange between 1×PBS and 5 mM Glucose, while FIG. 26B shows saturation voltages at different glucose steps.

FIGS. 27A-27B show in-vitro glucose step test of power transfer based smart hydrogel PI sensors. FIG. 27A shows results for solution exchange between 1×PBS and 12 mM Glucose with a 3 mM step, while FIG. 27B shows saturation voltages at different glucose steps.

FIGS. 28A-28B show in-vitro resetting test results for power transfer based smart hydrogel PI sensors. FIG. 28A shows results for solution exchange between 3 mM, 6 mM, and 9 mM glucose with a reset step of 1×PBS between each glucose step. FIG. 28B shows saturation voltages at different glucose steps.

FIGS. 29A-29C show results for in-vitro sensitivity for different steps of solution exchange measured as the ratio of change in voltage and change in solution concentrations. FIG. 29A shows results for the functionality test, FIG. 29B shows results for the step test, and FIG. 29C shows results for the resetting test.

### DETAILED DESCRIPTION

The present invention involves providing a power transfer-based flexible sensing platform or probe,that can be placed in a physiological fluid or other environment (e.g., the blood stream, attached to a cannula of a catheter, etc.), where monitoring of the concentration of an analyte within the fluid environment is desired. In a preferred embodiment, the device includes a hydrogel, sandwiched between two polymer-encapsulated metal layers at a distal tip of the device. The hydrogel swells or shrinks in response to the changing stimulus in the fluid environment, which changes the separation of the metallic layers with respect to each other. This change alters the signal transferred between the two metal layers. The metal structure at the tip of the device acts as a potentially directional antenna, and can be optimized by changing the directional characteristics, and thus how the signal coupling reacts to changes in separation distance and orientation changes induced by the hydrogel swelling or shrinkage. The tip may be configured in various ways, depending on the intended end use of the sensing platform. The polymer substrate simply encapsulates and carries the metal structure, and provides a point for attachment of the smart hydrogel. The geometry of the polymer substrate can be adapted to any intended application and sensing need, as long as the smart hydrogel can still alter the orientation or separation of the two encapsulated metal film layers that it is sandwiched between.

Another embodiment of the device includes a hydrogel, attached to a single polymer-encapsulated metal layer at a distal tip of the device. The hydrogel swells or shrinks in response to the changing stimulus in the fluid environment, which causes the polymer encapsulated metal layer to bend. This bending changes the impedimetric characteristics of the polymer embedded metal trace, which change can be read out, to determine the analyte concentration within the fluid.

### I. Introduction

Passive electrical circuitry-based flexible devices have been employed in various implantable sensor applications, e.g., ventricular artery pressure sensors, capacitive glucose sensors, flexible inductive transducers, and intraocular pressure sensors. Such devices are exposed to physiological fluids, whose electrical properties (conductivity and dielectric behavior) can lead to parasitic effects and introduce interference and artifacts. Mechanical deformation of the flexible circuit can also affect the electrical properties of the devices, especially in high-frequency applications. Such a mechanical deformation can either be caused inadvertently or introduced intentionally to be used as a sensor concept.

Sensor concepts based on intentional mechanical deformation of the electric circuit can, for example, employ hydrogels to introduce deformation. An example for this can be given by hydrogel-actuated movable diaphragm-based MEMS LC tanks that are used in radiofrequency (RF) sensing applications. While hydrogels in principle constitute a group of polymers containing a large amount of water (or other liquid), smart hydrogels are water-insoluble polymer networks which change their swelling state depending on external factors such as temperature, pH, light and analyte concentration. Hence, these polymers are of interest for sensor or actuator applications, especially in the biomedical context. Therefore, smart hydrogel structures can act as a sensing element, which responds to a change of analyte concentration/stimulus by swelling or shrinking. This can be used to cause mechanical deformation of the electric circuit sensor part it is attached to.

Such a deformation can be used to extract a sensor signal either by direct measurement of the electrical properties such as impedance of the sensor or, when two circuits are used to send and receive power/signals in an antenna-like fashion, by the change of the transferred power and/or signal such as described herein.

### II. Device Principle and Circuit Model

The presented investigation is based on a polyimide encapsulated metallic thin film bending sensor whose mechanical deformation is achieved by reversible swelling and shrinking of a smart hydrogel attached to one side (the tip) of the sensor.

. To better understand the device behavior and guide device design a lumped element circuit model was developed. To use the hydrogel as an actuator for sensor deformation, the device is often placed in a fluid environment. Therefore, in such a circuit model, the hydrogel's as well as the surrounding medium's parasitic influences have to be included as well as the electrical sensor properties. In the following, first, the device fabrication and operation principle of such simple deformation-based hydrogel sensors are briefly outlined. Based on this also a simple physics-based circuit model is presented.

### A. Principle of Operation and Fabrication of Flexible Polyimide Bending Sensor

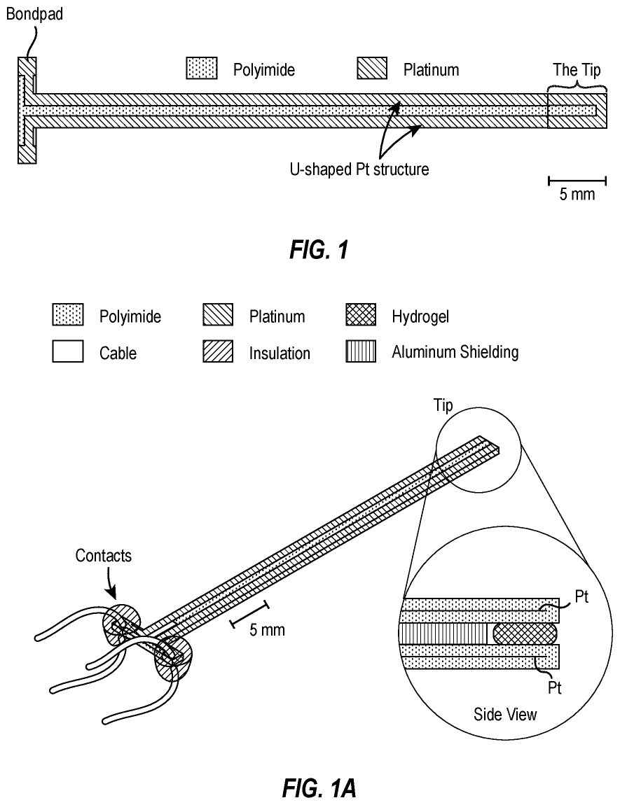

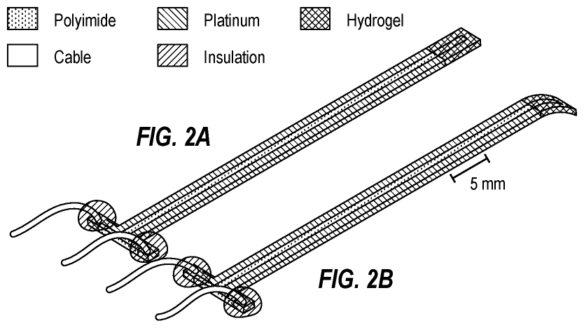

A simple one metallic layer sensor device includes a flexible current-carrying metal part encapsulated in polyimide and a smart hydrogel attached to one side of the flexible part's free end, as depicted in FIGS. 1 and 2A-2B. The attached hydrogel responds to a stimulus (e.g., ionic concentration within the fluid being monitored) by a reversible volume change. This volume change exerts a force on the thin, flexible polymer encapsulation and the embedded metal traces causing it to bend. The deformation of the conductive structure changes its impedance. With proper calibration, this change in impedance can be utilized to sense the ionic concentration of the medium.

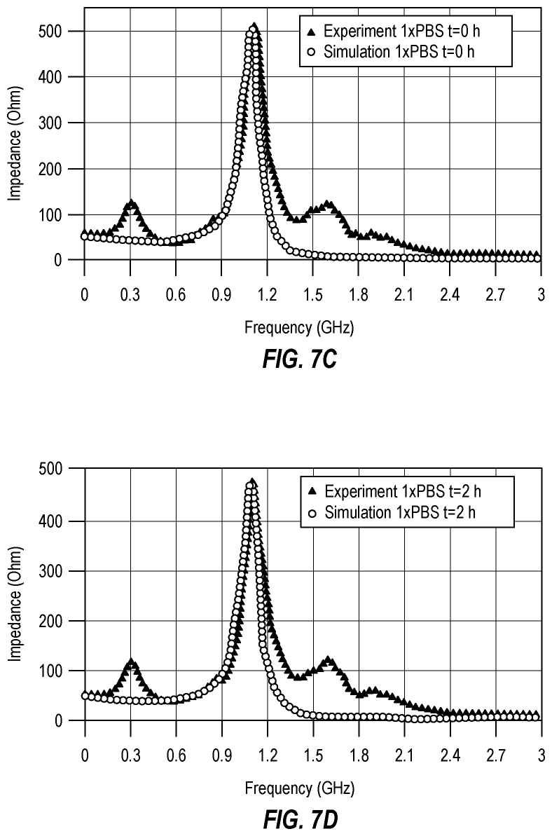

An exemplary sensor device may be prepared as follows: A 200 nm-thick and 0.975 mm wide U-shaped platinum structure with a 6.3 μm thick polyimide encapsulation was fabricated using spin-coating, lithographic pattern generation, sputtering, and/or lift-off techniques (see FIGS. 8A-8F for details).

Since the sensor is designed to be used in a biological environment (in the described in vitro experiments represented by phosphate-buffered saline (PBS)), the encapsulation protects the metal traces from the ions in the fluid. To electrically connect the sensor, the polyimide is etched at bond-pad locations away from the hydrogel-equipped tip using a lithographic technique to access the platinum layer and be able to connect wires to it.

Next, in the exemplary experiments an acrylamide-based smart hydrogel microstrip with ion sensitivity was attached to one side of the sensor tip with a chemically processed surface adhesion technique. For further details on hydrogel fabrication and attachment of this specific hydrogel, please refer to Nguyen et al., 2018, “Manipulation of the isoelectric point of polyampholytic smart hydrogels in order to increase the range and selectivity of continuous glucose sensors,” Sensors Actuators, B Chem., vol. 255, pp. 1057-1063, 2018, doi: 10.1016/j.snb.2017.08.022, which is herein incorporated by reference in its entirety. The dimensions of the hydrogel were 5 mm×2.7 mm×400 μm (width, length, and thickness, respectively).

Please note that the smart hydrogel in this case was chosen because of its ion sensitivity. If another analyte sensitivity is required, a different smart hydrogel can be chosen.

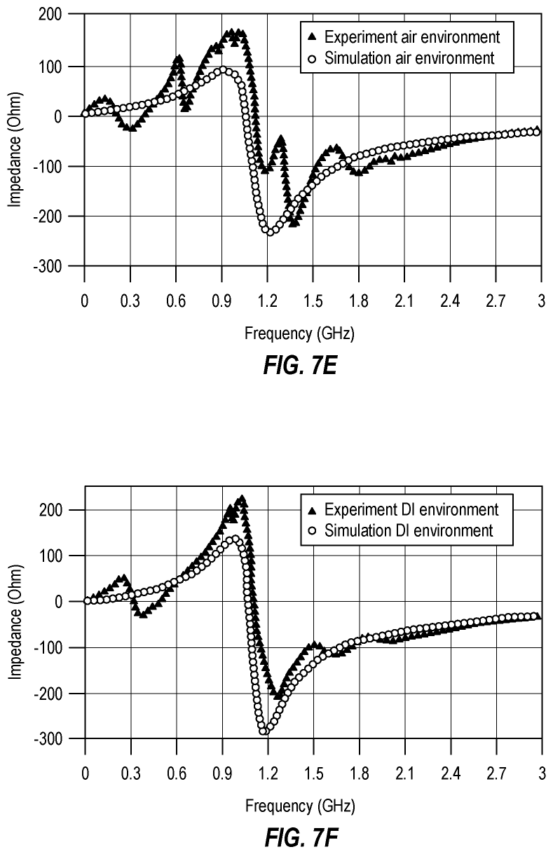

Please also note that the thickness of the polyimide and the hydrogel are chosen such that the polyimide can be bent or flexed reversibly and reliably by the hydrogel. The corresponding thickness relations between the two parts can be studied as described below, and details can be found in FIGS. 9A-9D.

While the 5 mm tip of the sensor was allowed to deform under the force exerted by the smart hydrogel during swelling and shrinking, the rest of the sensor portion was fixed to a polylactic acid (PLA) substrate of 45 mm length and 3 mm width with epoxy to avoid uncontrolled coiling and provide stability during the measurement.

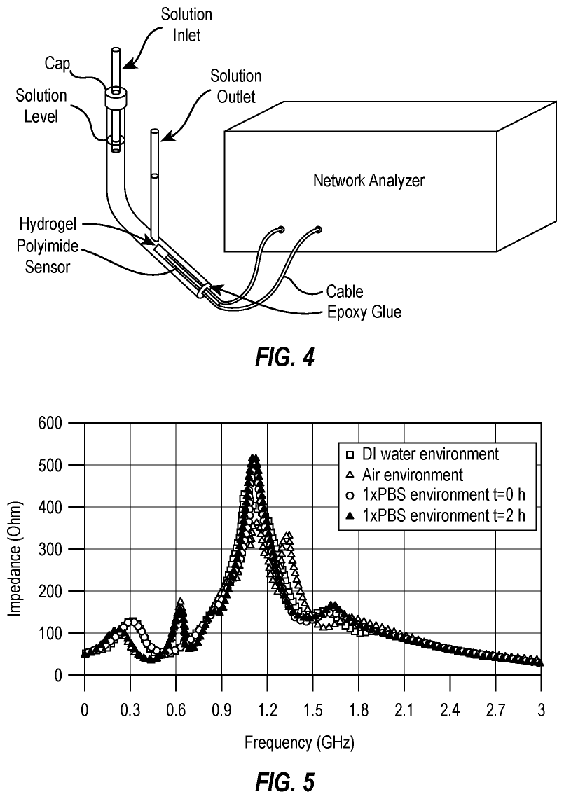

The sensor requires electrical connections for instrumentation which were achieved, in this specific case, by soldering co-axial cables to the bond-pads with connection points being encapsulated using a medical-grade epoxy to electrically insulate and mechanically protect them. However, since the epoxy becomes soft and degrades when exposed to a saline environment for prolonged periods of time, the sensor is designed in such a way that contact between the electrical connection points and the liquid environment in the employed experimental setup is avoided (see FIG. 4).

Hence, the dimensions of the employed exemplary final sensor were 50 mm×2.7 mm (length and width, respectively), of which only 40 mm of the sensor were inserted into the container with the liquid environment. Of course, in a device attached to a cannula for analyte detection and measurement in the blood stream, another bodily fluid stream, or other fluid, any electrical connection points may be formed to be durable in such an environment, or such connection points may be made “upstream”, outside of such an environment, as needed.

### B. Circuit Model

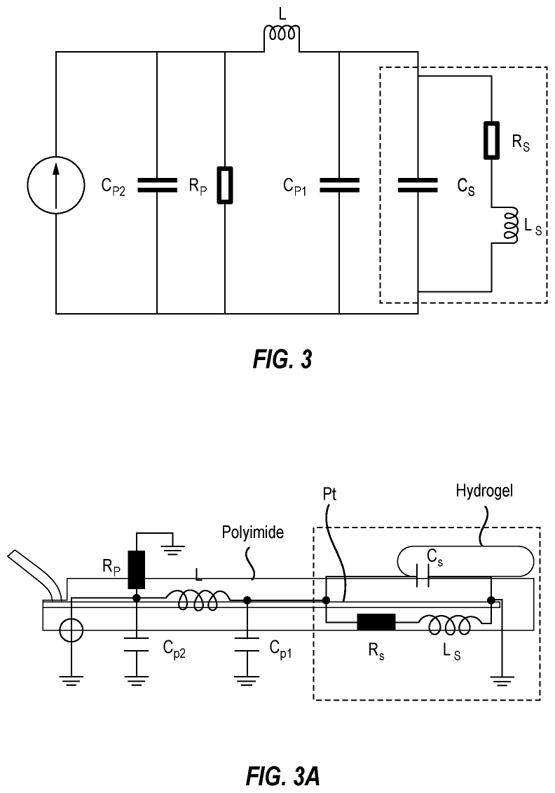

The flexible sensor system can be modeled as a basic inductor, as shown in FIG. 3 (framed part on the right, in dashed lines). It includes an inductor L_{S} (having inductance in Henries), a series resistor R_{S} (having resistance in Ohms), and a parallel capacitor C_{S} (having capacitance in Farads). However, the basic inductor model is not sufficient for reproducing the frequency behavior of the device as the surrounding environment exerts significant parasitic influences on the impedance spectra. In order to account for the connection wires, traces or cables, surrounding solution, and the shielding around the solution container, some elements need to be added to the circuit model. For example, an inductor L (having inductance in Henries), a parallel resistor R_{P} (having resistance in Ohms), and two parallel capacitors C_{P1} and C_{P2} (having capacitance in Farads), are introduced into the basic inductor model to take these into consideration. L and C_{P2} present the inductance and capacitance associated with the connector cable, wires, or traces between sensor and measurement equipment situated outside the experiment container (as shown in FIG. 4), and R_{P} represents the resistance at the transition between the sensor and the surrounding solution. C_{P1} denotes the sensor-to-ground shield capacitance and is a function of the medium material's permittivity and ionic properties.

In the complete equivalent circuit, L_{S}, C_{S}, R_{S} represent the self-inductance, the feedforward self-capacitance, and the series resistance of the sensor, respectively. This sensor is a single-turn thin film, so it does not have any turn-to-turn capacitance. C_{S} in this equivalent accounts for the fringing field in the air medium. To facilitate future sensor design and gain insights into the effects of the surrounding media and bending on the sensor signal, Applicant derived a lumped element circuit model. In contrast to existing approaches, which result in complex circuits, the present model was explicitly based on purely physical considerations and used as few elements as possible (see FIG. 3A).

### C. Experiment Design

The goal of the present experimental work was to study the effect of various surrounding media and mechanical bending on the sensor's frequency behavior. These results were then used as a basis for defining the circuit elements of the respective model and studying their influences on the sensor response. Three different environments were considered: air, deionized (DI) water, and phosphate-buffered saline (PBS) solution.

### Introduction of Different Surrounding Media

The first impedance measurement was carried out in air, and then DI water was introduced in the container through the inlet of the solution container, applying positive pressure. Application of negative pressure at the outlet was avoided as that might generate a vacuum inside the container and deform the flexible sensor. The solution container was tilted at a 90° angle during the introduction of DI water to ensure that no bubbles are generated around the sensor. However, to exchange the solution from DI water to 1×PBS, the inlet was connected to a reservoir of 1×PBS solution, and negative pressure was applied at the outlet, as complete removal of DI water from the container before the introduction of 1×PBS deforms the flexible tip due to surface tension. In order to ensure complete exchange of the liquid volume, 50 mL solution (10 times the volume of the solution container) was withdrawn through the outlet.

### D. Impedance Measurement Procedure for the Flexible Sensor

The impedance measurements of the sensor were performed with a Keysight E5061B Network Analyzer and a co-axial cable with a BNC port which was connected to the network analyzer with a BNC to N-port adapter. During all measurements, the aluminum shield on the container was connected to the network analyzer ground to avoid floating potentials. The instrument was calibrated to the co-axial cable end with an 85032F Type N calibration kit. The measured spectra were recorded in .txt format for 5 Hz to 3 GHz frequency range with 1601 linear sampling points for each spectrum.

The experiment was designed to investigate the impact of the dielectric and conductive properties of the surrounding medium as well as the bending deformation on the impedance characteristics of the sensor in order to derive the parameters of the circuit model and also shows the functionality of the sensor. To quantify the effect of dielectricity of the medium, first the impedance spectra of the sensor were measured in two media with distinctly different dielectric constants, air, and DI water. Next, the DI water was replaced with 1×PBS which maintains the dielectric properties of the medium similar to DI water but changes the medium's ionic concentration. An impedance spectrum was measured in 1×PBS solution immediately and again two hours after introducing the solution.

The hydrogel changes its swelling state in the PBS environment (shrinking compared to DI water) which in turn bends the sensor. The measured impedance spectrum after the introduction of the 1×PBS solution gives the impact of conductivity of the medium on the sensor, while the impedance spectrum taken after two hours gives the impact of sensor deformation.

### III. Results

### A. Experimental Impedance Measurements

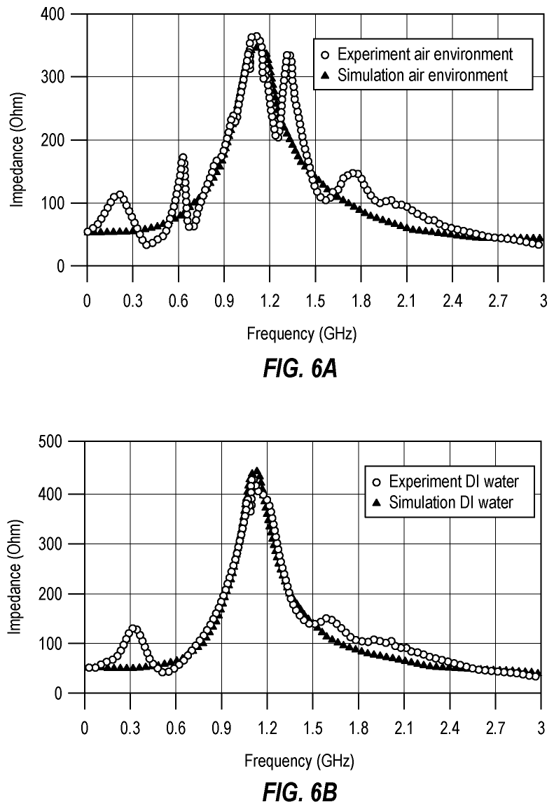

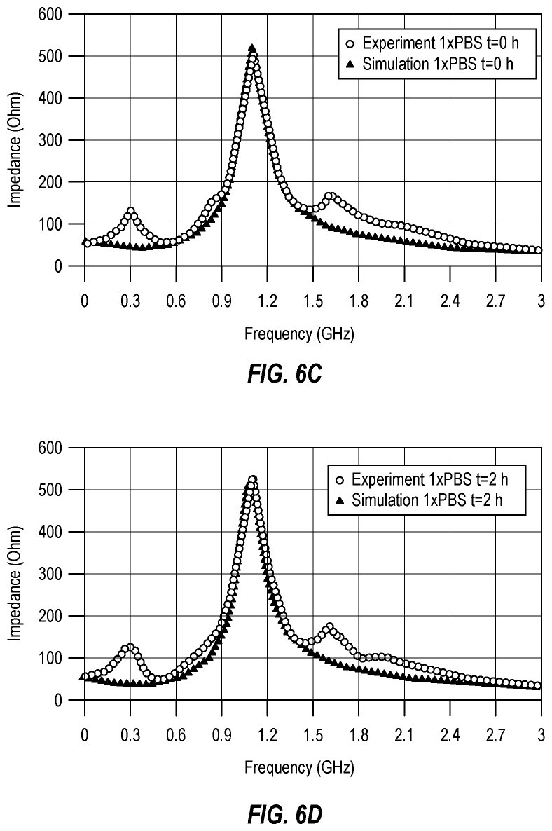

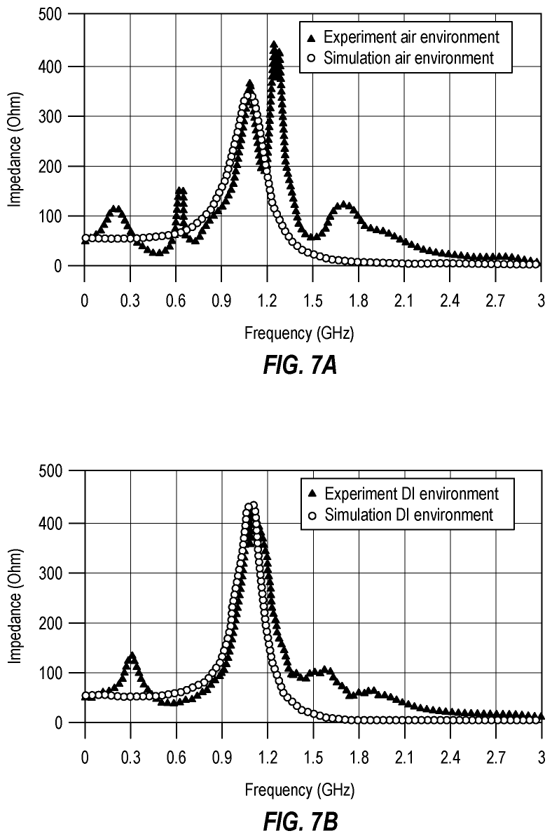

The impedance spectra of the sensor were measured experimentally in different conditions with a Network Analyzer as described above. FIG. 5 shows the sensor's experimentally obtained impedance magnitude in air, DI water, and in the PBS environment right after solution introduction and after hydrogel saturation (exposure for two hours, i.e., completed sensor bending) for a frequency range from 5 Hz to 3 GHz.

Several resonance peaks can be observed for all media with one main resonance peak, except in the case of air, where two peaks of similar impedance magnitude are present. However, due to the behavior of the dominant peaks through the medium exchange, it was assumed that the left peak of the air spectrum is the relevant one. While these experiments were aimed at better understanding the functional principle of the sensors and deriving the parameters for the lumped model, the change in the impedance spectra can also be read out by measuring the sensors impedance at a fixed frequency, ideally on or close to the main resonance peak.

### IV. Further Work with Glucose Sensitivity, Using Dual Layer Sensors

Earlier systems providing a sensor platform where hydrogel swelling causes deformation of a thin film with embedded metallic structures, resulting in an impedance change, can exhibit problems of high noise, stability, and reproducibility. The presented configurations, which provide for inductive power transfer between two thin metal film layers or parts with the hydrogel sandwiched in between, provide for better stability, increased sensitivity, significantly reduced noise and enables reproducible measurements. Furthermore, to predict sensor behavior for the design process, Applicant has derived a simplified electric circuit model which allows direct evaluation of the effects of surrounding media and hydrogel-induced sensor bending on the output signal. An overview of the key aspects of this novel concept for sensitive detection of a smart hydrogel's volume change are provided herein.

The sensor in this example was based on two thin film components, each including a U-shaped platinum layer (400 nm thickness) encapsulated in 6.3 μm thick polyimide. Both parts have electrical contact pads on one end and are arranged in parallel with an aluminum shield in between (e.g., such as depicted in the inset of FIG. 1A). In the shield-free tip region (e.g., 5 mm length) a smart hydrogel of desired thickness is polymerized between the two thin films. The hydrogel dimensions can be adjusted depending on the target application. The proof-of-principle sensors were 50 mm long and 2.76 mm wide with a 380 μm thick hydrogel on the tip. The two polyimide thin films with the embedded U-shaped platinum layer were arranged in parallel in close proximity with an aluminum shield around and in between, with the hydrogel sandwiched at the tip. The surrounding aluminum shield is not shown in FIG. 1A for better visibility of the sensor.

The transduction of the hydrogel's volume change into an electric signal is based on inductive power and/or signal transfer mediated by electromagnetic fields between the two thin film metal components. One acts as a transmitter with an applied sinusoidal signal and the other acts as a receiver for the induced voltage. Swelling and shrinking of the hydrogel alters the distance and/or angle between the two thin films, leading to a distinct change of the transmitted energy and hence, induced measurement voltage.

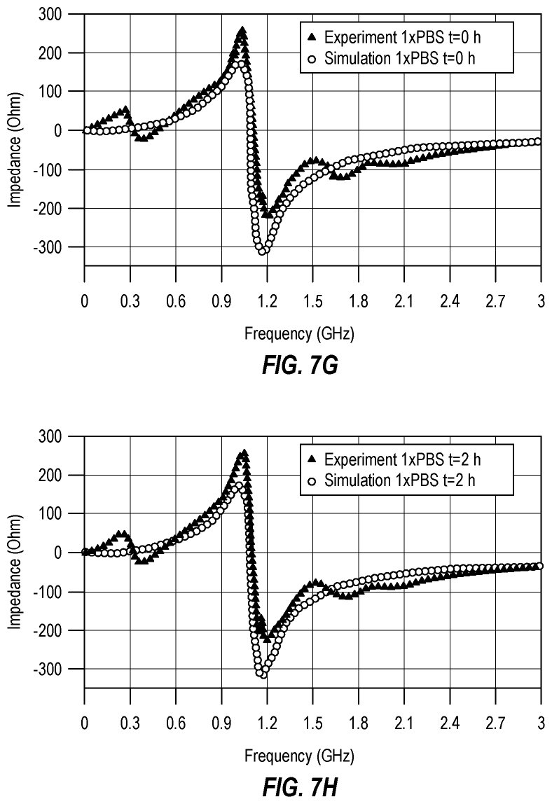

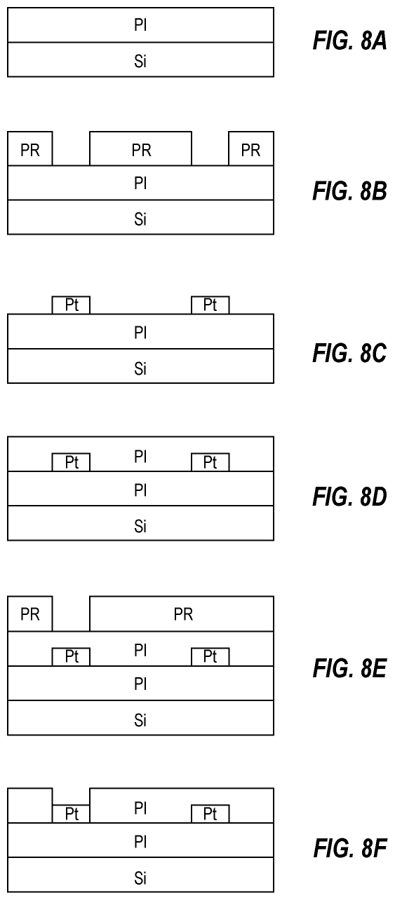

To evaluate sensor behavior, Applicant employed a glucose-sensitive polyacrylamide hydrogel which responds to changes in ion concentration and glucose in the mMol range. The sensor has been tested in alternating ion concentrations, as well as different glucose concentrations to study repeatability, stability and sensitivity. FIG. 10 depicts an exemplary result for alternating 0 mM and 5 mM glucose response (both in phosphate buffered saline (PBS) as carrier solution) which clearly indicates good reproducibility.

The presented inductive power transfer-based detection concept enables robust and sensitive detection of smart hydrogel volume changes, with potentially biocompatible components. Moreover, a physics-based circuit model facilitates sensor design and prediction of sensor performance for specific use cases. Hence, this is an important step in expanding the potential of smart polymers for future biomedical analyte detection.

### V. Further Work With Hydrogel Thickness and Encapsulating Substrate Thickness

Further work was also conducted further study and testing relative to the link between hydrogel thickness and substrate thickness for sensors as described herein.

One major challenge for using smart hydrogels for purposes as described herein is the development of suitable transduction mechanisms for the polymer's volume change. In addition to the ability for reliable detection of swelling changes of a usually transparent or slightly opaque hydrogel, such a transducer should ideally be biocompatible for biomedical applications.

Several mechanical deformation-based concepts based on silicon technology-based pressure sensors and cantilevered structures have been described in literature. However, while these approaches can provide high sensitivity especially in the case of cantilever transducers, many of these concepts face challenges with respect to biocompatibility due to being made from silicon or other materials that may not provide good biocompatibility, or they require relatively large read-out schemes and thus space requirements. In an embodiment, any portion of the device implanted within the body does not include silicon, or any other non-biocompatible material.

The Applicant has developed potentially biocompatible transducers based on the deformation of metal traces which can be equipped with any type of smart hydrogel. Hence, it provides a platform which is easily adaptable to different sensing contexts. In order to tailor the sensor for specific applications, the transducer needs to be designed accordingly which requires insights into the interdependence between deformation behavior and hydrogel properties. In the following working example, a parameter study of geometric properties (smart hydrogel and polyimide thickness as well as spring constant) is presented as well as their coherences and implications on sensor bending in order to guide and facilitate future sensor design. Furthermore, a 50-cycle bending test of a mechanical dummy sensor structure is presented to evaluate the mechanical stability.

In order to investigate the interdependencies of mechanical properties for application specific sensor design the bending stiffness of the transducer with respect to hydrogel actuation force was studied. For this a mechanical sensor dummy structure comprising only the polyimide film and hydrogel, was considered. The influence of the metal traces with a thickness of a few hundred nanometers on the stiffness of the several micrometers thick polyimide film was deemed negligible and thus the metal traces were not included in the mechanical dummy. This allows one to directly study the influence of polyimide geometry and hydrogel thickness on the resulting amount of bending.

For the included study, rectangular polyimide sheets (length: 7.1 mm, width: 3.5 mm) were fabricated with varying thicknesses (1.4, 3.6, 6.3, 12.0 and 17.2 μm).

The spring constant k of these polyimide sheets can then be calculated based on their dimensions and material properties using equation (1).

$$k = \frac{E \cdot W \cdot {T}^{3}}{4 \cdot {L}^{3}} \qquad ( 1 )$$

E denotes the encapsulating polymer's Young's modulus, W, T and L the width, thickness and length of the sheets respectively. The Young's modulus was measured experimentally with a nanonindenter instrument (Hysitron TI Premier) to obtain the presented results.

Subsequently, polyacrylamide-based smart hydrogels (thicknesses: 124, 240 and 400 μm) were attached to one side of the polyimide sheets. For this, the sheets were first treated chemically and then the smart hydrogels were deposited with a photo-polymerization method employing molds. In the resulting samples the hydrogels cover two thirds of the sheet length and the full sheet width. These samples where then affixed to a holder that allows for free bending of the portion with the hydrogel.

To measure the magnitude of mechanical bending, the samples were immersed in two different concentrations (⅓× and 1×) of phosphate buffered saline (PBS). The first measurement was carried out after an initial resting period in 1×PBS (24 h). Then after each solution exchange the measurement was carried out after a wait time of 30 minutes to allow the hydrogel's volume change to reach its equilibrium state. The bending angle change was recorded using a digital optical microscope and evaluated using an image analysis software.

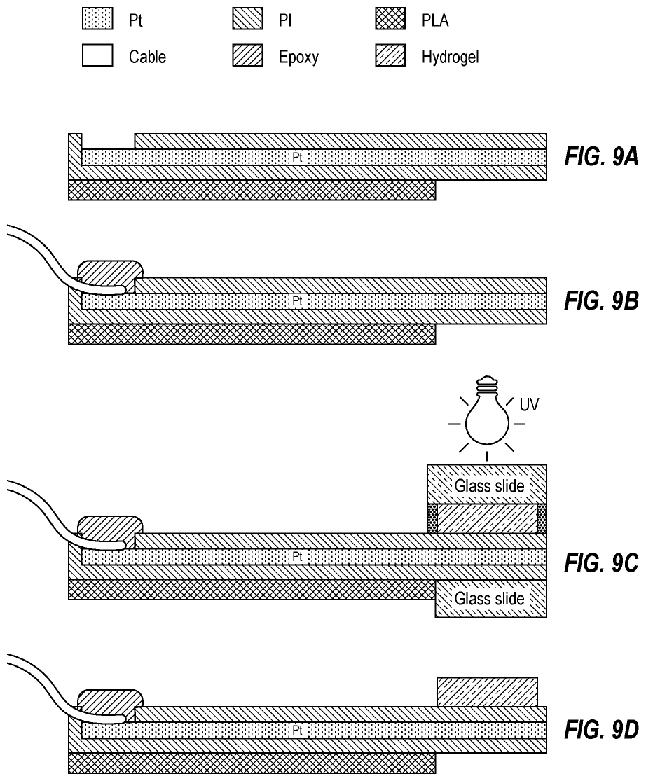

FIG. 12A depicts the calculated spring constants for each polyimide thickness and FIG. 12B depicts the resulting bending angle change in two different PBS concentrations for three hydrogel thicknesses in relation to the polyimide spring constants.

Additionally, as repeatability is an important property of sensor devices, the bending stability was studied on a 6.3 μm thick polyimide sheet with a 400 μm thick smart hydrogel, by changing the PBS concentration between the two values 100 times and obtaining the resulting bending angles as described above.

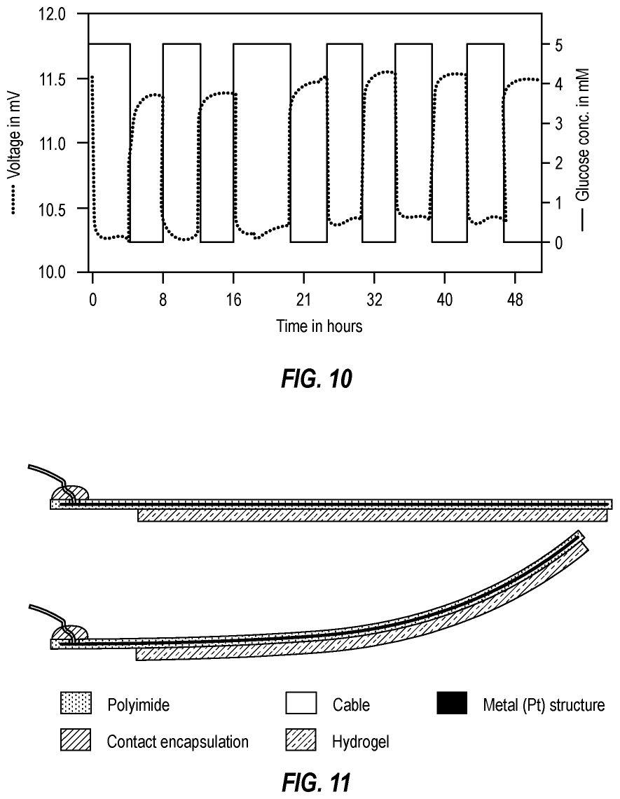

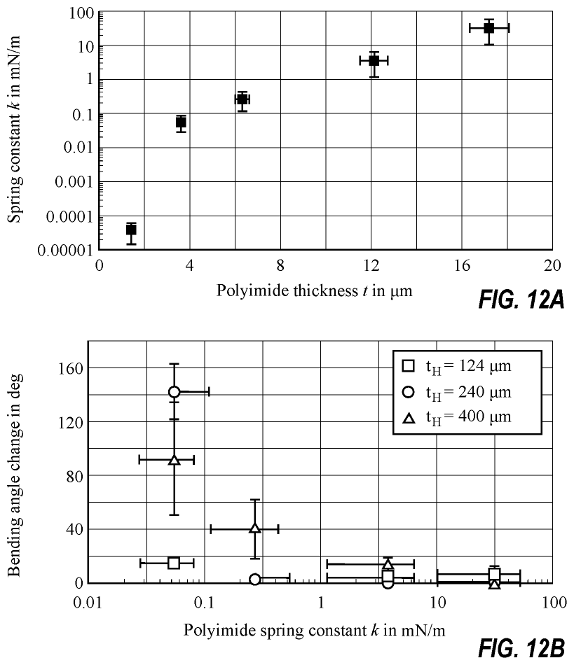

The resulting bending angles and their change over time are depicted in FIGS. 13A-13B.

The obtained results can be used to study the achievable bending of a single-layer of polymer with a smart hydrogel actuator for a given set of geometric parameters with respect to sensor design. For this several factors need to be considered: the amount of mechanical resistance of the polymer substrate against deformation by the forces acting upon it as well as the force exerted by the smart hydrogel sensing element.

While the mechanical resistance of the substrate mainly depends on its stiffness, the maximal achievable actuation force depends on the amount of volume change but also on the hydrogel's dimensions. The volume change of a smart hydrogel is influenced by a variety of parameters, such as the specific stimulus, hydrogel material and internal polymer structure. The hydrogel's dimensions are important parameter for sensor design as there are several tradeoffs included. A large piece of hydrogel might exhibit a stronger absolute volume change and thus excerpt a larger force on the substrate. However, as the volume change of a smart hydrogel often depends on the diffusion of an analyte into its polymer network, an increased volume can lead to longer response times of the sensor. Additionally, if the hydrogel bends the substrate, at some point the structure will obstruct itself as its surfaces will start to touch each other.

When considering the interdependence of hydrogel thickness vs. polyimide stiffness, a balance needs to be found. The transducer should exhibit a significant bending change within the expected range of hydrogel volume change. A too thin polyimide film (polymer encapsulation) will result in a too strong deformation, reaching mechanical limits such as self-constriction of the structure as explained above when the hydrogel is not fully swollen/shrunken thus leading to early saturation of the sensor's signal. Additionally, damage to the polymer encapsulation is possible.

In Applicant's experiments furthermore twisting (e.g., longitudinal axial twisting) of the polyimide observed instead of bending was observed when the film was too thin in relation to the hydrogel thickness. This is the reason why there is no data set for the thinnest polyimide sheet (1.4 μm) in FIG. 12B. Thus, in an embodiment, the sheet may have a thickness of greater than 1.5 μm, greater than 2 μm, or greater than 3 μm (e.g., from 3 to 10 μm).

Another aspect of this bending concept is the stability, i.e. the repeatability when the sensor is subject to cyclic swelling and shrinking. As shown in FIGS. 13A-13B for an exemplary dummy structure, the bending angles as well as the change thereof are reduced over time. A likely explanation is given by a degradation of the adhesion between polymer substrate and smart hydrogel due to repeated stress at the contact surface due to the bending. This will need to be addressed when designing a sensor product by updated fabrication procedures to improve the longevity of the sensor.

Based on this example it is shown how one would approach obtaining a suitable hydrogel thickness for any kind of polymer encapsulation shape and hydrogel configuration, facilitating future sensor development.

### VI. Working Example Showing Use of Sensor with pH Sensitive Hydrogel

A flexible sensor platform to transduce the volumetric transition of a hydrogel when exposed to specific analytes is described above. Previously this concept was evaluated with a glucose sensitive hydrogel (GSH), as has been reported above. Nevertheless, this concept can be applied to any type of responsive hydrogel, allowing it to be adjusted to a particular purpose. In order to validate this claim, we conducted similar experiments with a pH-sensitive hydrogel as were done with GSH. This aims to present a working example that the presented sensor technology is a platform that can be equipped with different smart hydrogels to obtain sensors for different analytes.

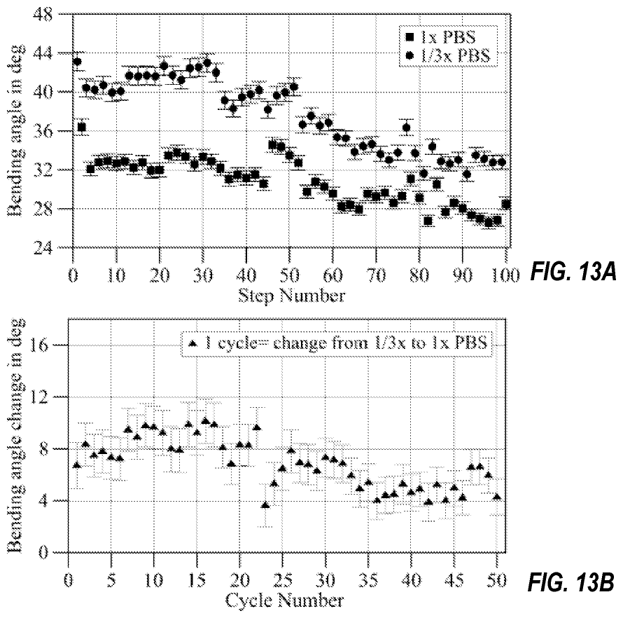

The sensor used in this working example included, as described previously, two U-shaped conductive platinum structures which are encapsulated by non-conductive PI films, as shown in FIGS. 1A and 16, having dimensions as shown in FIG. 15. The polyimide is etched at one end to access bond pads (platinum contact) for cable connection. The PI sheets are connected back-to-back with an aluminum sheet in between. The aluminum film works as a shield to reduce crosstalk between the platinum structures. The 5 mm portion on the opposite end of the polyimide is referred to as ‘the tip’ (as shown in FIG. 16), and does not include the aluminum shielding.

A pH sensitive smart hydrogel is attached in a sandwiched configuration between the PI films at the flexible tip. There the hydrogel works as an actuator to bend the PI and the metallic structure when responding to an external stimulus. In order to introduce the power transfer, one of the platinum structures is energized, and the power or signal transferred to the other structure is measured. The swelling or shrinking of the hydrogel changes the separation distance and/or angle between the thin metallic encapsulated structures, i.e., altering the effective area for power transfer. Hence, power transfer between the metal layers at the tip is dependent on the swelling of the hydrogel. When properly calibrated, this electric signal can be used to quantify the stimulus. For this working example, we use wires to interface the sensor with the measurement instrument (Zurich Instrument UFHLI Lock-in amplifier). For a future application, this could be substituted by an integrated wireless system for remote readout.

### Sensor Structure Design

Each metallic thin film structure was designed as a simple U-shaped loop to reduce the current path (see FIGS. 15-16), i.e., the thin film resistance, in order to enhance current, which is crucial for power transfer efficiency. Furthermore, it keeps the metallic structures very simple which facilitates the fabrication process and can enhance the lifetime of the thin metallic traces. While an increase in the number of turns might enhance the power or signal transfer between the structures, it would also increase the current path, which in turn leads to greater thin-film resistance, i.e. reduced current and thus potentially reduced signal strength and signal-to-noise ratio. In order to improve the signal-to-noise ratio, metal shielding was used (as shown in FIG. 16) to cover the sensor, except for the tip.

### Results

### 1. Short Power/Signal Transfer-Based Sensor

Two experiments were conducted to validate the sensor concept. Initially, the short sensor (15 mm in length as shown in FIG. 15) was tested to measure the changes in solution concentration between pH 7.4 and 7.7 in 1×PBS solution environment and the results were documented with a lock-in amplifier. To further assess the response of the sensor in a wider range a step test was performed which involved a gradual increase and decrease of the pH level from 7.4 to 8.2 and back to 7.4, with a 0.2 pH step height. This step test was done twice to validate reproducibility of the sensor behavior.

### A. Functionality Test

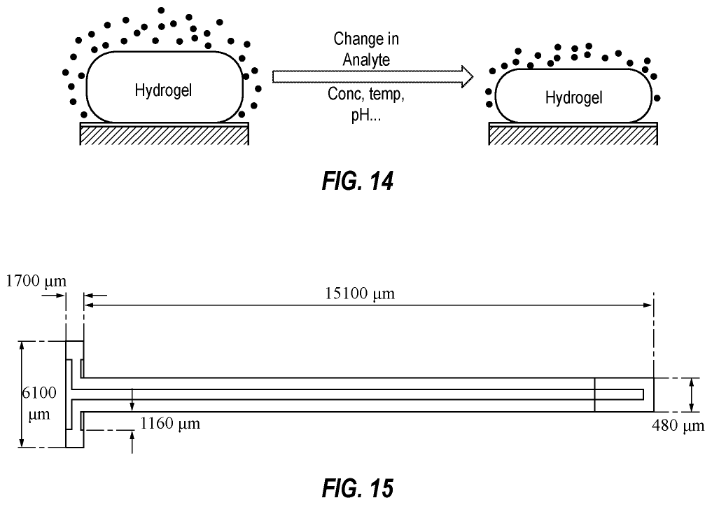

To evaluate the performance, the sensor was first put through a basic functionality test. After filling the sensor container with a 1×PBS solution of pH 7.4, the solution was left stagnant and unchanged for 24 hours. Subsequently, the solutions were exchanged using a syringe pump between 1×PBS of pH 7.4 and 7.7 for 6 cycles with an interval of 6 hours in between (FIG. 17).

### B. Step Test

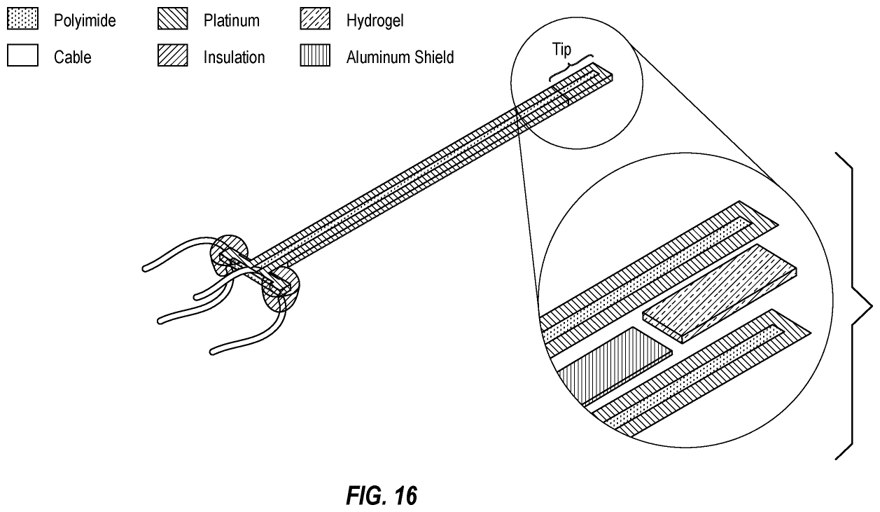

We tested the step response of the sensors to different pH by first immersing them in 1×PBS solution of pH 7.4 for 24 hours to ensure saturation of hydrogel volume change. Then, we sequentially exchanged the solution to 1×PBS solutions of increasing pH levels (7.6, 7.8, 8.0, and 8.2) at 6 hour intervals, before returning to the initial pH level of 7.4. This cycle was repeated twice to evaluate the sensor response's repeatability. Results are shown in FIG. 18.

### 2. Long Power/Signal Transfer-Based Sensor

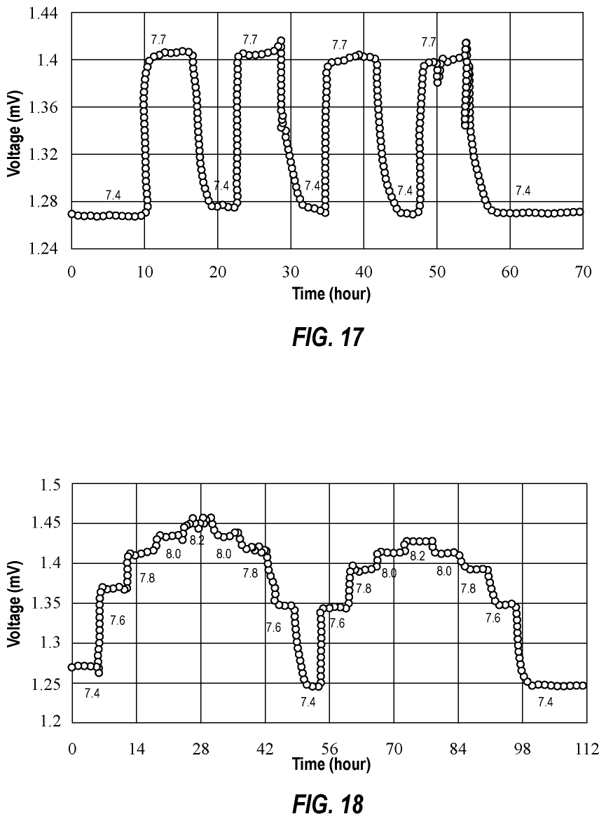

A similar study was conducted using sensors made of glucose-sensitive hydrogel that were longer than the pH-sensitive hydrogel sensors previously. The length of the conductive structure was set to 50 mm and the width was kept at 480 μm, which was the same width as the shorter sensors. FIG. 19 schematically illustrates the longer sensor, with dimensions provided in micrometers.

### A. Functionality Test

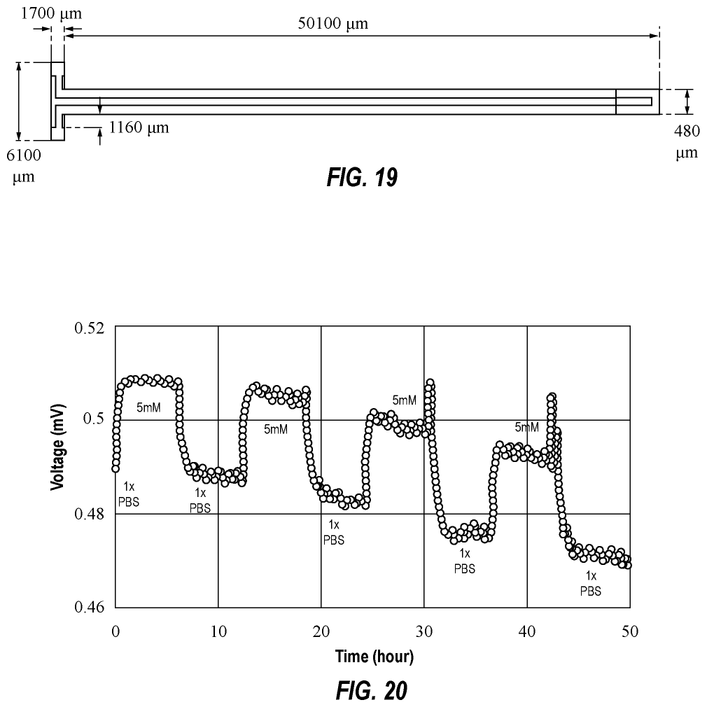

To assess the basic performance of the sensor, the sensor container was initially filled with 1×PBS solution, and left undisturbed for 24 hours. Subsequently, a syringe pump was used to exchange the solution with a 5 mM glucose solution, and this cycle continued 8 times with a 6-hour interval between each exchange (see the sensor response in FIG. 20).

### B. Step Test

Although a step test was performed, no usable results were obtained. This illustrates that the impedance of the metal structure needs to be taken into account and optimized, as a too high impedance of the structure was found to be detrimental to signal strength and quality.

### C. Reset Test

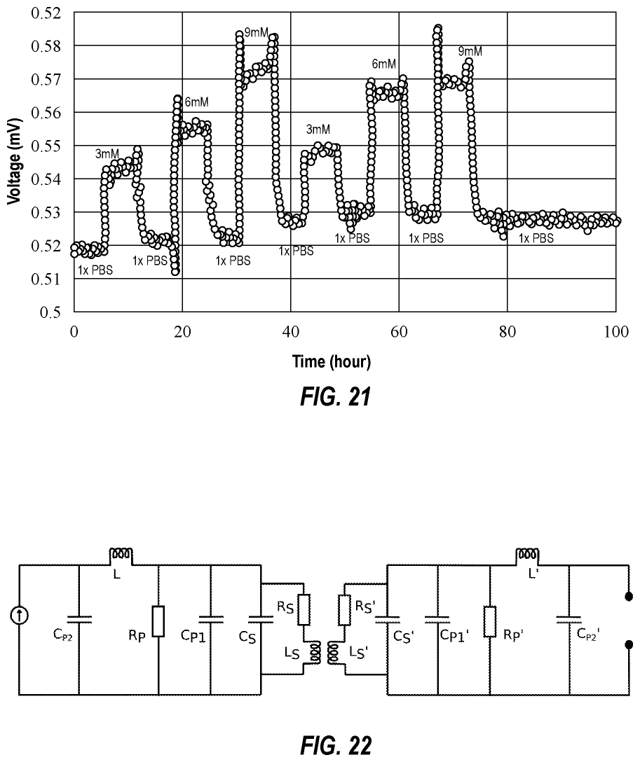

A reset test was performed to measure the sensor's behavior when it is exposed to 1× PBS from different glucose concentrations. We put the sensor in 1×PBS for 24 hours, then switched to 3 mM, 6 mM, and 9 mM glucose solutions in succession, and reset back to 1×PBS for each step. We did this twice to make sure the results were consistent. The results are shown in FIG. 21.

### Preliminary Circuit Modeling

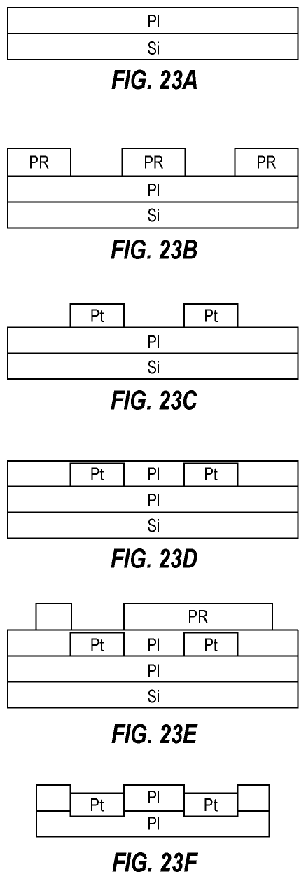

A circuit model for a power/signal transfer based smart hydrogel PI sensor, which is an improvement of the hydrogel based impedimetric bending sensor is being developed. The circuit model will utilize the knowledge gained from the previous model. This circuit model is shown in FIG. 22.

In the circuit model, L_{S}, R_{S}, and C_{S} represent the inductance, resistance, and capacitance of the sensor, C_{P1} is the sensor to ground capacitance, L, and C_{P2} are the cable inductance and capacitance. R_{P} is the sensor to solution resistance for the side which is connected to the power source. L_{S}′, R_{S}′, and C_{S}′ represent the inductance, resistance, and capacitance of the sensor; C_{P1}′ is the sensor to ground capacitance, L, and C_{P2}′ are the cable inductance and capacitance, and R_{P}′ is the sensor to solution resistance for the readout side.

### CONCLUSION

The short sensor (15 mm in length) has been demonstrated to be capable of responding to varying pH levels in solutions, as well as glucose sensitive hydrogel. This suggests that the sensor is not specific to any particular smart hydrogel, but rather can be used as a general platform for different smart hydrogels.

The long sensor, which was 50 mm in length, exhibits a gradual decline (drift) in its signal over time as well as a lack of response during the step test, believed to be due to a decrease in sensitivity caused by the impedance of the conductive structure increasing with its length. Construction of a circuit model for this sensor platform will allow optimization of the sensor design. This circuit model will allow us to anticipate the performance of future versions of the sensor for both in-vitro and in-vivo applications and guide further sensor improvements and development.

### VII. Additional Working Example

In this working example, we developed a smart hydrogel-interlayered flexible sensor for biomarker monitoring utilizing the power transfer capabilities between two PI-encapsulated electrically conductive structures. Inductive power transfer (IPT) couples the power from a transmitter track or coil to a receiver coil where both the transmitter and the receiver are tuned to the same frequency to enhance power transfer. IPT has found applications in power systems, biomedical implants, automation, electric vehicles, and instrumentation. A hydrogel-based RF resonator sensor is reported which utilizes the change of capacitance between two split rings to modulate the resonant frequency. However, this sensing technique reads the frequency shift due to the change of capacitive coupling at a very high frequency (>500 MHz). Unlike capacitively coupled systems, here we present a sensing system that counts on the change of inductively transferred power to readout the change of the swelling state of a smart hydrogel at a low frequency (e.g., ˜30 MHz).

The proposed power transfer-based sensor platform can be applicable for both in-vitro and in-vivo applications. For in-vivo applications, the active circuitry for readout can be implemented in-vitro while only the sensing node needs to be implanted. As explained previously, the sensor features the following main advantages: the sensor is made of biocompatible materials, is very thin and flexible, causing a reduced foreign body response and potentially allowing for various applications in different media. Furthermore, the sensor is easily adaptable for different analytes as the smart hydrogel can be tailored as needed without influencing the readout of the swelling state.

### A. Sensor Concept

In inductive power transfer (IPT), power is transferred between two current-conducting loops by a magnetic field, where the transmitter and the receiver conductors form a transformer. As described in Ampere's law, an alternating current (AC) through the transmitter loop generates an alternating magnetic field. The magnetic fluxes pass through the receiver and induce an electromotive force (EMF) which causes an AC current in the receiver. The magnitude of the induced EMF, and hence the AC current amplitude depends on the separation between the transmitter and the receiver. The smart hydrogel positioned in between such loops modulates the distance between the transmitter and the receiver, such that the separation between the loops can be read out from the amplitude of the induced EMF or the current amplitude.

In this working example, we utilized a smart hydrogel as the modulating agent to alter the separation between the transmitter and the receiver. In between a transmitter and a receiver, we introduced a thin layer of hydrogel which changes its volume in response to different stimulus, i.e., changes the separation between the transmitter and the receiver, i.e., the induced current. The change in the induced current can be calibrated to determine the amount of stimulus. In this example, a glucose sensitive hydrogel (GSH) is used for evaluation purposes; however, the concept can be employed with any smart hydrogel, making it easily adjustable for a specific application.

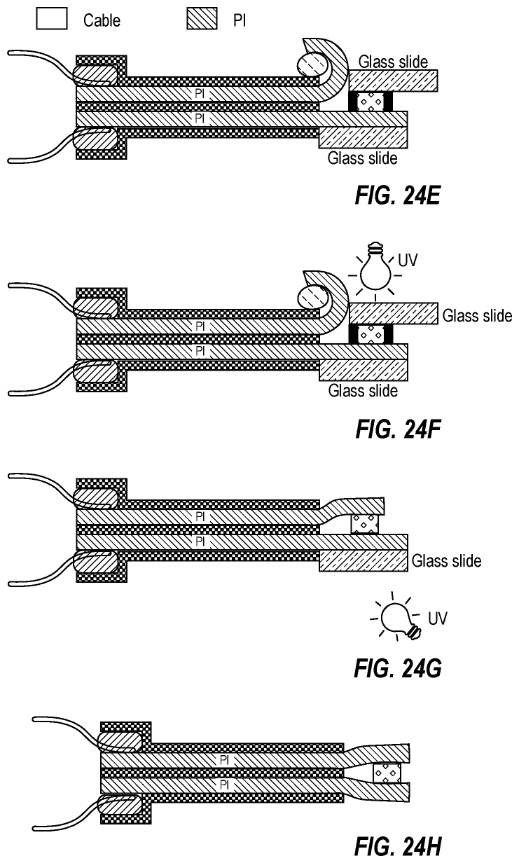

The current sensor included two 50 mm long and 2.7 mm wide U-shaped conductive platinum structures encapsulated by 6.3 μm thick non-conductive PI films, as shown in FIG. 16. The thickness, trace width, and the distance between the traces of each of the platinum structures were 400 nm, 0.975 mm and 0.7 mm, respectively. Each PI sheet was etched at one end to allow access to the bondpads on the platinum structure which facilitates cable connection for instrumentation. A 45 mm portion of the PI sheets were glued back-to-back with an aluminum sheet in between (refer to FIGS. 24A-24H for details) except for the 5 mm long ‘tip’ which is located on the opposite end relative to the bondpad (as shown in FIG. 16). The glued portion of the PI film remains mechanically stable and prevents coiling of that portion while the tip remains flexible. The aluminum sheet works as an electromagnetic shield to reduce crosstalk between the platinum structures.

In order to create electrical connection between the sensor and the measuring instrument, co-axial cables RG-174 are connected to the bondpads. The bondpad-cable junction is sealed with medical grade epoxy which serves two purposes: mechanical reinforcement and insulation to the contact point.

A 5 mm×2.7 mm×400 μm (length, width, and thickness, respectively) GSH was attached between the PI films at the flexible tips which works as an actuator to alter the separation between the back-to-back connected PI and the conductive structures at the tip when responding to an external stimulus. One of the platinum structures of the sensor was connected to the power source, and the transferred power to the other U-shaped structure was measured. The swelling or shrinking of the GSH changes the separation distance between the tips of such platinum structures. Hence, power transfer between the tips is dependent on the swelling of the GSH. When properly calibrated, this electrical signal can be used to quantify the stimulus. In the working example, wires were used to interface the sensor with the measurement instrument (Zurich Instrument UFHLI Lock-in amplifier) for proof of principle experiments. For future applications, this could be substituted by an integrated wireless system enabling remote readout.

### B. Materials and Method

As discussed above, the sensor is composed of two PIs encapsulated platinum structures with aluminum shielding, a smart hydrogel, contact cables, and an insulating adhesive. In the following, the sensor microfabrication along with the hydrogel interlayer attachment technique utilizing the pre-gel solution and the experimental plan to characterize the sensor are discussed.

### (i). Fabrication

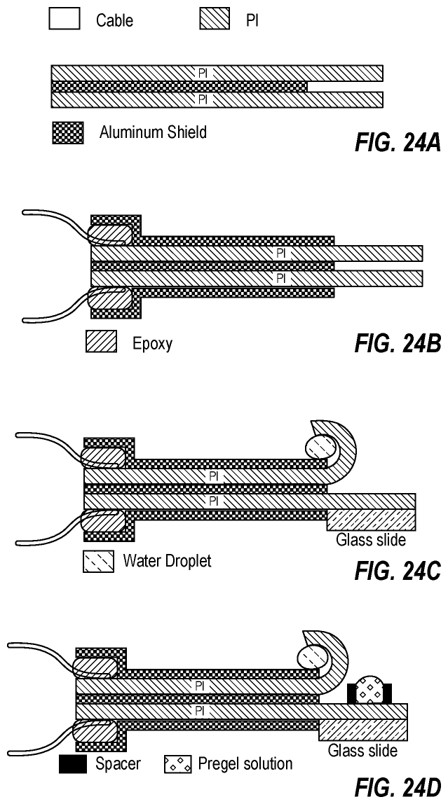

The complete power transfer-based sensor was fabricated in two steps: first, the individual PI encapsulated metal structures were prepared by microfabrication (see FIGS. 23A-23F), and then two of these were assembled with a smart hydrogel sensing element and shielding in between, as shown in FIGS. 24A-24H. First, liquid polyimide was spin-coated on a silicon carrier wafer, followed by a soft baking and a hard baking step (FIG. 23A). Then the PI film was patterned with standard lithography steps (lift-off resist, e.g., positive tone photoresist, and developer) to create U-shaped structures (FIG. 23B). Pt/Ti was deposited by sputtering, and the conductive U-shaped structures were generated by a subsequent lift-off of the photoresist (FIG. 23C). Then, another layer of polyimide was spin-coated and baked for encapsulation of the metallic traces (FIG. 23D). The outline of the sensor structures and the bond pads were then created by a second lithography step (e.g., with photoresist and developer). A deep reactive ion etching (DRIE) process with CF₄, O₂, and N₂ gases was then performed using the photoresist as the etch mask to remove polyimide between the sensors, as shown in FIGS. 23E and 23F. Finally, the carrier wafer with sensors was rinsed with acetone and deionized (DI) water to remove the photoresist residue.

The microfabricated PI films were peeled off from the silicon carrier wafer individually with tweezers, and two of them were connected back-to-back with epoxy adhesive with an aluminum shield in between, as outlined in FIG. 24A. Cables were connected to the bond pads by soldering, and then the connection points were protected with epoxy. Aluminum shields were then attached to the other side of the PI films to reduce the impact of interfering signals, as shown in FIG. 24B.

Next, the GSH layer was attached between the PI tips in a sandwich configuration using a UV polymerization technique. In order to control the thickness of the hydrogel, polymerization was carried out in two steps. First, the hydrogel was polymerized on the lower PI sheet while the other sheet was bent by employing the adhesive force of a water droplet to avoid UV absorption (see FIG. 24C). A glass slide was placed underneath the lower sheet to stabilize and keep it flat during the process. The pre-gel solution of the hydrogel was placed in a 380 μm thick polytetrafluoroethylene (PTFE) mold, and a second glass slide on top of the mold ensures the desired thickness of the hydrogel (see FIG. 24D). After UV polymerization of the hydrogel (FIG. 24F), the water droplet was removed to release the bent PI.

The sensor was then washed in DI water to remove any unpolymerized pre-gel solution. Then, 1 μL pre-gel solution was brushed on the other PI surface with a pipette tip. Next, the PI was pressed gently on the hydrogel, and UV light exposure from both sides and the top ensures good attachment of the PI surface with the hydrogel (FIG. 24G). Finally, the glass slide under the PI surface was removed (FIG. 24H), and the sensor was again washed thoroughly with DI water to remove any unpolymerized solution.

### (ii). Experiment Design

The experiments were designed to observe the response of the sensor in a phosphate-buffered saline (PBS) environment at different glucose concentrations. The sensor characterization experiments were performed in a continuous flow setup while the sensor was contained in a small-channel of 135 mm³ (45 mm×3 mm×1 mm length, width, and height respectively) volume to ensure quick exchange of solution concentrations. In order to get only the sensor response avoiding any external interference, the channel was covered with an electromagnetic shield and the sensor response was recorded with the help of a lock-in amplifier. The experimental details are discussed in the following sub-sections.

### Experimental Setup

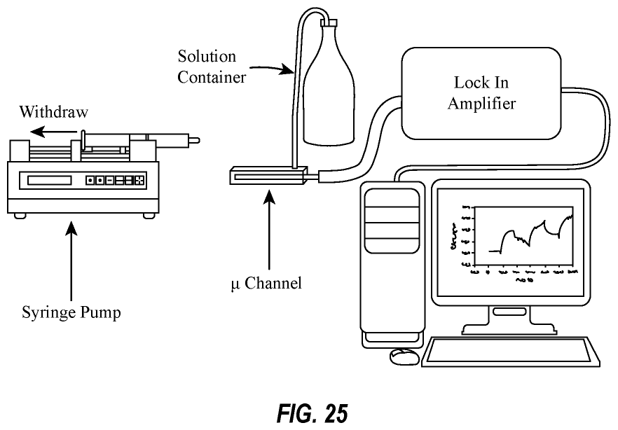

The experimental setup includes the sensor, a channel with an inlet and an outlet, solution container bottles, a lock-in amplifier, and a computer for data recording (as shown in FIG. 25). The sensor was inserted into the channel. Please note that a 40 mm portion from the tip of the 50 mm long sensor was placed inside the channel while the rest of the sensor and the connector cables were placed outside. The channel was then sealed with medical-grade epoxy adhesive and then silicone gel after insertion of the sensor. The inlet of the channel was connected to a solution container bottle while the outlet was connected to a syringe pump with silicone tubing. The sensor cables were connected to 50Ω BNC female connectors which were connected to the lock-in amplifier with the help of 50Ω coaxial cables with BNC-male ends. The lock-in amplifier was connected to a computer for data recording.

### External Noise Suppression

In order to observe only the sensor behavior in response to different glucose concentrations, other influences need to be suppressed. Although the junction is encapsulated by medical-grade epoxy, the quality of the encapsulation degrades with time in the PBS environment. Hence, the 40 mm portion of the 50 mm long sensor was placed in a small-channel (experimental container). The soldered bondpads and connection cables were placed outside of the container, as the sensor response might drift with the degradation of the epoxy on the bondpad.

The channel was wrapped with an aluminum shield which is further connected to the shield of the connector cables, i.e., the ground shield of the measuring instrument (lock-in amplifier) to avoid any external interfering signal. Furthermore, the cables were taped tight on the experiment bench to avoid any vibration-induced sudden change in the signal.

### Solution Exchange Procedure

The sensor and the surrounding solution concentration needs to be stable to ensure a stable initial point. Hence, the channel containing the sensor was filled with 1×PBS solution and kept unaltered for more than 24 hours to ensure saturation of the hydrogel swelling. Then the analyte solutions were exchanged between 1×PBS and different concentrations (depending on the experiment protocol as shown in Table 1) of glucose with a four-hour interval to observe consistency and repeatability of the sensor response. Note that glucose solutions of different concentrations were prepared in 1×PBS solution. For example, a 5 mM glucose solution indicates 1×PBS+5 mM glucose solution.

In all experiments, solutions were exchanged utilizing the syringe pump with a continuous flow of 0.1 mL/min flow rate. However, switching the inlet from one solution container to another might introduce bubbles to the channel. Hence, the syringe pump was stopped first, and then the container was changed, and then the syringe pump was started again.

TABLE 1
Solution exchange protocol for different experiments

| Solution | Functionality | | |
|---|---|---|---|
| Step | Test | Step Test | Resetting Test |
| initial | 1× PBS | 1× PBS | 1× PBS |
| 1 | 5 mM | 3 mM Glucose | 3 mM Glucose |
| | Glucose | | |
| 2 | 1× PBS | 6 mM Glucose | 1× PBS |
| 3 | 5 mM | 9 mM Glucose | 6 mM Glucose |
| | Glucose | | |
| 4 | 1× PBS | 12 mM Glucose | 1× PBS |
| 5 | 5 mM | 9 mM Glucose | 9 mM Glucose |
| | Glucose | | |
| 6 | 1× PBS | 6 mM Glucose | 1× PBS |
| 7 | 5 mM | 3 mM Glucose | 3 mM Glucose |
| | Glucose | | |
| 8 | 1× PBS | 1× PBS | 1× PBS |
| 9 | 5 mM | 3 mM Glucose | 6 mM Glucose |
| | Glucose | | |
| 10 | — | 6 mM Glucose | 1× PBS |
| 11 | — | 9 mM Glucose | 9 mM Glucose |
| 12 | — | 12 mM Glucose | 1× PBS |
| 13 | — | 9 mM Glucose | — |
| 14 | — | 6 mM Glucose | — |
| 15 | — | 3 mM Glucose | — |
| 16 | — | 1× PBS | — |

### Data Recording

Time-dependent responses of the power transfer-based sensor were recorded with a lock-in amplifier using different concentrations of glucose and 1×PBS. The voltage change due to power transfer was recorded with a sampling frequency of 1.67 samples/sec at 30 MHz operating frequency with a third-order Lock-in amplifier internal filter. However, this frequency was chosen based on the frequency spectrum at different solutions. In order to choose the frequency, first, a frequency spectrum is recorded right after the introduction of 5 mM glucose solution and then again after 4 hours when the hydrogel is saturated. Then the operating frequency was chosen where the sensor presents a considerable amount of change. The transmitter input signal amplitude was chosen to be 100 mV_{P}.

### C. Results

In order to validate the sensor concept, three experiments were performed with three different solution exchange protocols. First, a basic functionality test on the sensor was performed with the solution concentration exchanges between 0 mM and 5 mM glucose, and the sensor response was recorded with the help of a lock-in amplifier. In order to better assess the sensitivity, next, the sensor response was studied for stepping up of glucose concentration from 0 mM to 12 mM and then stepping down to 0 mM with a solution exchange step of 3 mM. This experimental protocol is referred to as step test and was performed for two cycles. Finally, a resetting test was performed on the sensor in order to assess the repeatability of the sensor. This test protocol includes exchange of concentrations among 3 mM, 6 mM, and 9 mM glucose with a reset step to 0 mM between each glucose concentration step. The solution exchange protocol for three different experiments are provided in Table 1. The solution exchange interval between each concentration is 4 hours for all three experiments.

### Functionality Test

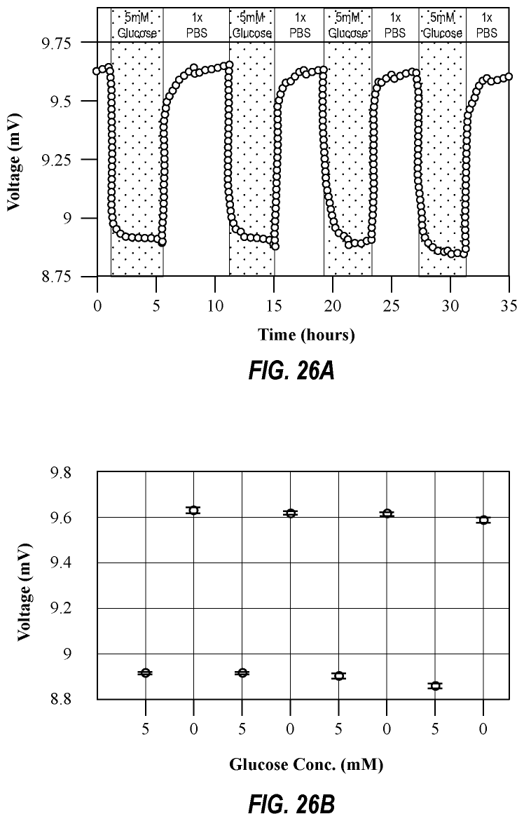

For the basic functionality test, the channel containing the sensor was first filled with PBS solution of 1× concentration, and the solution was kept stagnant and unaltered for 24 hours. Then the solution was exchanged with 5 mM glucose solution, with the help of the syringe pump, and the solution exchange continued between 1×PBS and 5 mM glucose for 8 cycles with a solution exchange interval of 4 hours (FIG. 26A).

The sensor showed repeated response with an average 9.65 mV saturation voltage in 1× PBS solution and 8.91 mV saturation voltage in 5 mM glucose with an average standard deviation of 0.0061 mV (1×PBS) and 0.0062 mV (5 mM glucose), respectively (refer to FIG. 26B). Note that the saturation voltage is calculated from the average of the last 30 minutes voltage of each solution exchange interval and the error bars indicate the first standard deviation of the saturation voltage. For the other two experiments, i.e., step test, and resetting test, the saturation voltage and the error bars were calculated in the same manner.

### Step Test

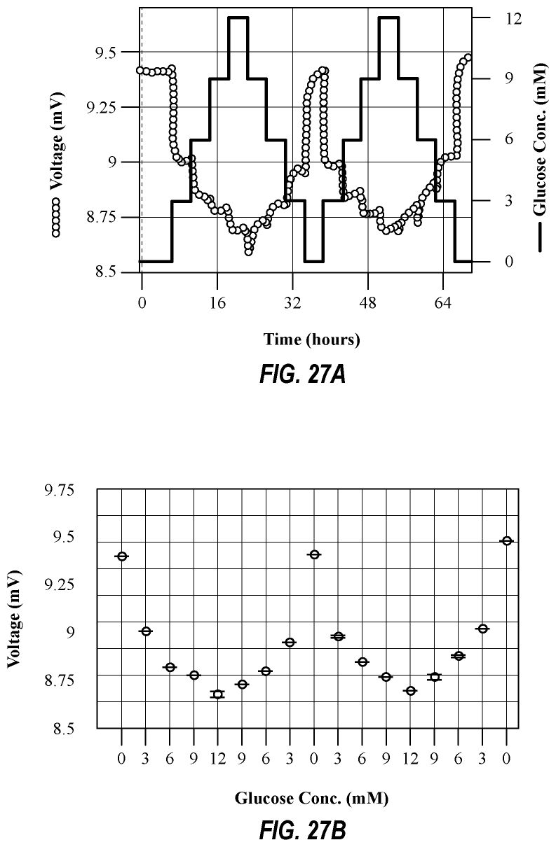

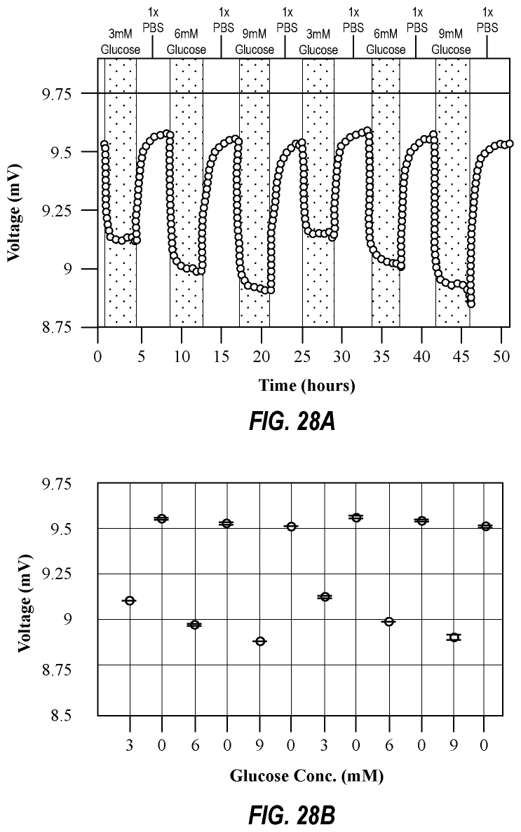

Next, we performed studies on the step response of the sensors to different concentrations of glucose (FIG. 27A). For the step response test, the sensor was kept in 1×PBS solution for 24 hours, same as the functionality test, to ensure the complete saturation of hydrogel volume change. Then solutions 3 mM, 6 mM, 9 mM, and 12 mM glucose were introduced with 4 hours of exchange interval and then cycled back to 1×PBS with the same 3 mM glucose step. The complete cycle continued twice to observe the repeatability of the step response.

With the increment of glucose concentration, the sensor showed a reduction in the sensor response, and showed an inversion in the response with the solution exchange from 9 mM to 12 mM glucose. The magnitude of the sensor response to different concentrations of glucose indicates the sensor voltage magnitude can be indicative of the glucose concentration from 0 mM to 9 mM glucose.

### Resetting Test

Next, we performed a reset test on the sensor in order to observe the sensor response for sudden reset to 0 mM glucose, i.e., 1×PBS from different concentrations. The sensor was first introduced to 1×PBS in the beginning of the experiment and kept in the solution for 24 hours. Then the solution was exchanged to 3 mM glucose, 6 mM glucose and 9 mM glucose sequentially. However, between each glucose step, the solution was reset back to the initial 1×PBS state. This complete test was performed for two cycles to ensure repeatability of the test.

### Sensitivity Analysis

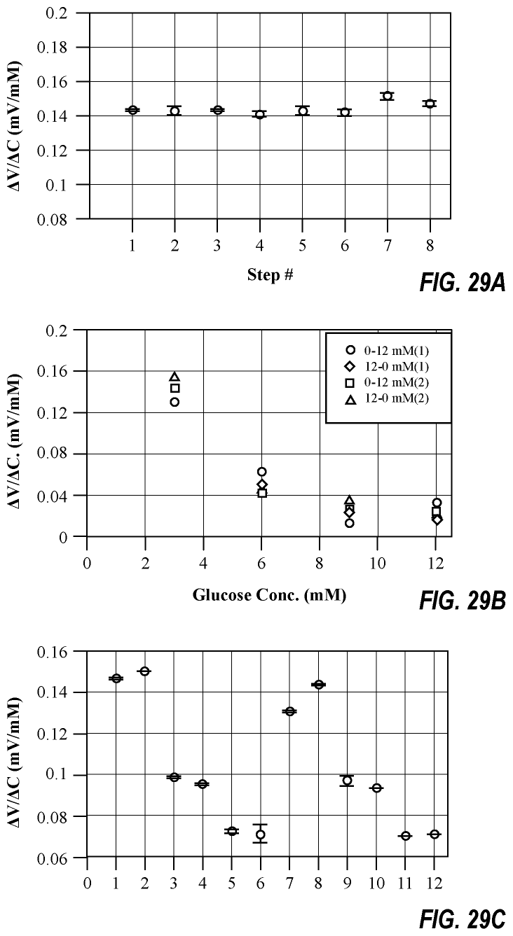

Furthermore, we performed a sensitivity analysis on the sensor response during the above-mentioned three experiments (FIGS. 29A-29C). The sensor exhibits a consistent sensitivity with an average of 0.142±0.005 mV/mM during the functionality test. The step test shows a somewhat consistent sensitivity during the first cycle while the second cycle showed reduced sensitivity. The resetting test also shows high sensitivity in solution exchange between 1× PBS and 3 mM glucose. However, the sensitivity decreases at 1×PBS to 6 mM solution exchange and further reduces in 1×PBS to 9 mM glucose step.

### D. Discussion

This working example aims to develop a generic biocompatible sensor platform to transduce the response of a smart hydrogel to different stimuli into an electrical signal for both in-vitro and in-vivo applications. For this purpose, a power transfer-based sensing platform to facilitate continuous transduction of hydrogel's volumetric state change is introduced. However, a phenylboronic acid based GSH as a smart hydrogel model is chosen for this study which shrinks/swells with the increase/decrease in glucose concentration in the surrounding aqueous media, as binding of the PBA with glucose molecule increases the water potential of the hydrogel. However, the GSH in the sensor platform can be replaced with any hydrogel which changes its volume in response to different stimuli. We used a GSH only for demonstration of the proof of principle of the sensor platform.

The sensor comprises two PI encapsulated thin metallic conductive structures with hydrogel interlayered flexible tips. One of the conductive structures is connected to a power source while the induced voltage at the other structure is measured. The volume change of the GSH changes the separation of the flexible conductive structures and hence alters inductively transfers power between the structures which is then read out as a change in voltage magnitude.

The metallic structure is designed as a simple U-shaped loop to reduce the current path, i.e., the thin film resistance, in order to enhance current which is crucial for power transfer efficiency.

The response of the sensor is read out with a lock-in amplifier. We experimented with PBS medium which poses a very high ionic concentration (˜145 mM) close to the ionic concentration of blood. As the sensor can perform well in a high ionic environment, we anticipate that the sensor can be utilized in blood and other fluids such as saliva, sweat, or urine which poses a moderate ionic concentration (˜20 mM).

The GSH used for this study also shows the cross response to a change in pH and ionic concentration. The experiment is designed to perform in a 1×PBS environment while the solution pH is maintained at 7.4 which is the same as the blood pH.

The working example is designed to run a series of experiments (functionality test, step test, resetting test) to observe the repeatability of the sensor response in different conditions. The results show that the sensor offers a consistent response from 0 mM to 12 mM glucose range during the step test. The resetting test shows a slight shift in the signal baseline, indicating glucose becomes complexed to the PBA in the GSH. An improvement in the hydrogel chemistry would resolve this issue.

It is also observed that the shrinking response of the hydrogel is twice as fast as the swelling response. This phenomenon can be explained as swelling of the hydrogel requires pushing the PI sheets which is similar to compressing a spring while the shrinking is facilitated by the PI sheet which can be compared to relaxing a compressed spring.

One potential failure mode for this sensor is the delamination of hydrogel from the PI surface. In order to prevent delamination, first, we treated the PI surface with oxygen plasma to improve adhesion. In addition, we further treated the surface chemically with methanol, aminopropylmethacrylamide·HCl, and distilled tributylamine for improved adhesion.

It should to be noted that several phenomena need to be considered to perform similar in vivo studies. As the sensor needs to be implanted, the soft tissue around the sensor might mechanically restrict the volumetric change of hydrogel and the flexibility of the PI films. If this issue occurs in the future in-vivo experiments, then the sensor can be covered with a permeable membrane or mesh which will allow the physiological fluid to contact the hydrogel and prevent the tissue from being in direct contact with the sensor.

The analysis of the sensitivity of the sensor response shows a very consistent behavior in all three experiments (functionality test, step test, and resetting test). The step test and resetting test show that the sensor presents a somewhat reduced response in high glucose concentration which agrees with other studies on phenylboronic acid-based GSH. This can be attributed to the saturation of volumetric change of hydrogel.

In summary, we performed three experiments on the power transfer-based sensing platform and observed reproducible responses from all the tests. The sensor showed a non-linear response at high glucose-concentration which agrees well with previous studies on the hydrogel. The experimental results provide proof of principle of the functionality of the sensor, the range of application for a specific type of hydrogel. This experimental success can be transferred to different types of hydrogels and in-vivo studies in the future.

Any of the embodiments as described herein may include an enclosure (potentially with electrical shielding), with a membrane around the tip part, enclosing with the hydrogel within such membrane. This can provide at least two potential advantages
(i) if the membrane allows the analyte in question to pass through (e.g., permeable or semi-permeable membrane), this might serve to prevent damage to the hydrogel and catch pieces of the hydrogel even if they break off during use. This would prevent these pieces from entering the blood stream or other fluid stream, when used as a catheter-based sensor. Such a configuration could prevent potential negative effects that a hydrogel might have such an induced coagulation, while still allowing the sensor to function as described herein;
(ii) a membrane can be used that can transduce the analyte concentration outside the membrane, to another analyte concentration inside the membrane, which could make the sensor even more versatile; if an analyte concentration is for example transduced into a pH change, this could be picked up by a pH sensing smart hydrogel already employed as described herein.

As used herein, the term “between” includes any referenced endpoints. For example, “between 2 and 10” includes both 2 and 10.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

Disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure. Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A power and/or signal transfer-based flexible sensing platform used in a fluid environment comprising:
at least one metallic thin film structure,
the metallic thin film structure having a polymeric encapsulation that protects the metallic thin film structure from the fluid environment;
a smart hydrogel sandwiched between layers of the metallic thin film structure, the smart hydrogel being configured to swell or shrink in response to stimulus; and
a power source electrically connected to the at least one metallic film structure.

2. The sensing platform as recited in claim 1, wherein the layers of the metallic thin film structure are electrically shielded from one another, at a location apart from the smart hydrogel sandwiched therebetween.

3. The sensing platform as recited in claim 1, wherein the fluid environment is a physiological fluid environment.

4. The sensing platform as recited in claim 1, wherein the stimulus that the smart hydrogel swells or shrinks in response to comprises at least one of pH, temperature, or concentration of an analyte.

5. The sensing platform as recited in claim 1, wherein the stimulus that the smart hydrogel swells or shrinks in response to is an analyte concentration, which analyte is present in the fluid environment.

6. The sensing platform as recited in claim 5, wherein the analyte comprises at least one of pH or glucose concentration of the fluid in the fluid environment.

7. The sensing platform as recited in claim 5, wherein the analyte comprises concentration of a drug in the fluid environment.

8. The sensing platform as recited in claim 1, wherein the metallic thin film structure comprises a noble metal.

9. The sensing platform as recited in claim 1, wherein the metallic thin film structure comprises platinum.

10. The sensing platform as recited in claim 1, wherein the polymeric encapsulation comprises a polyimide.

11. The sensing platform as recited in claim 1, wherein the hydrogel is functionalized with one or more chemical moieties, an analyte-specific aptamer or molecular imprinting to be sensitive to a specifically selected analyte.

12. The sensing platform as recited in claim 1, wherein the smart hydrogel is present at a distal tip of the sensing platform, the sensing platform further comprising an enclosure with a membrane around the distal tip including the smart hydrogel.

13. The sensing platform as recited in claim 12, wherein the enclosure includes electrical shielding.

14. The sensing platform as recited in claim 12, wherein the membrane is configured to transduce an analyte concentration outside the membrane, to another analyte concentration inside the membrane.

15. The sensing platform as recited in claim 14, wherein the sensing platform transduces an analyte concentration in the fluid environment outside of the enclosure to a pH change within the enclosure, the smart hydrogel inside the enclosure being configured to swell or shrink in response to the change in pH.

16. The sensing platform as recited in claim 1, wherein the power source applies an alternating current to the at least one metallic film structure.

17. The sensing platform as recited in claim 16, wherein the AC current has a frequency of 5 to 100 MHz.

18. The sensing platform as recited in claim 1, wherein the at least one metallic thin film structure comprises a pair of U-shaped metallic thin film structures, each encapsulated in a polymeric encapsulation, wherein the smart hydrogel is sandwiched between the pair of U-shaped metallic thin film structures, the smart hydrogel being configured to swell or shrink in response to the stimulus, wherein the power source is electrically connected to a first U-shaped metallic thin film structure of the pair of U-shaped metallic thin film structures, so as to cause power to be transferred from the first U-shaped metallic thin film structure to a second U-shaped metallic thin film structure of the pair of U-shaped metallic thin film structures by induction when the power source is activated, measurement of the induced power in the second-U-shaped metallic thin film structure being used to determine a concentration of an analyte in the fluid environment.

19. A power and/or signal transfer-based flexible sensing platform used in a physiological fluid environment comprising:
at least one metallic thin film structure,
the metallic thin film structure having a biocompatible polymeric encapsulation that protects the metallic thin film structure from the physiological fluid;
a smart hydrogel sandwiched between layers of the metallic thin film structure, the smart hydrogel being configured to swell or shrink in response to stimulus;
a power source electrically connected to the at least one metallic film structure; and
read out electronic or other equipment that is electrically connected to the at least one metallic film structure, for read out.

20. A impedimetric flexible sensing platform used in a fluid environment comprising:
at least one metallic thin film structure,
the metallic thin film structure having a polymeric encapsulation that protects the metallic thin film structure from the fluid environment;
a smart hydrogel attached to the metallic thin film structure, the smart hydrogel being configured to swell or shrink in response to stimulus, so as to cause the metallic thin film structure to bend at a different angle when the stimulus is present, versus when the stimulus is not present; and
a power source electrically connected to the at least one metallic film structure.
